SEQUENCE LISTING (14 sequences)

SEQ ID NO: 1 - protein, 37 aa, synthetic construct
  VARIANT 16..25: note = Any amino acid
  VARIANT 16..25: note = This region may encompass 1-10 residues or may be absent
SYEKSHSQAINTDRTXXXXXXXXXXEQLGVRKELRGV

SEQ ID NO: 2 - protein, 5 aa, synthetic construct
EAAAK

SEQ ID NO: 3-4 - skipped (2 entries)

SEQ ID NO: 5 - protein, 5 aa, synthetic construct
PAPAP

SEQ ID NO: 6 - protein, 5 aa, synthetic construct
GSGGG

SEQ ID NO: 7 - protein, 12 aa, synthetic construct
EQLGVRKELRGV

SEQ ID NO: 8 - protein, 15 aa, synthetic construct
SYEKSHSQAINTDRT

SEQ ID NO: 9 - protein, 32 aa, synthetic construct
SYEKSHSQAINTDRTEAAAKEQLGVRKELRGV

SEQ ID NO: 10 - protein, 27 aa, synthetic construct
SYEKSHSQAINTDRTEQLGVRKELRGV

SEQ ID NO: 11 - protein, 30 aa, synthetic construct
SYEKSHSQAINTDRTAPAEQLGVRKELRGV

SEQ ID NO: 12 - protein, 30 aa, synthetic construct
SYEKSHSQAINTDRTGGGEQLGVRKELRGV

SEQ ID NO: 13 - protein, 32 aa, synthetic construct
SYEKSHSQAINTDRTPAPAPEQLGVRKELRGV

SEQ ID NO: 14 - protein, 32 aa, synthetic construct
SYEKSHSQAINTDRTGSGGGEQLGVRKELRGV

(19) United States
(12) Patent Application Publication
(10) Pub. No.: US 2025/0213452 A1
(43) Pub. Date: Jul. 3, 2025

(54) BIFUNCTIONAL PEPTIDES FOR BIOMIMETIC RECONSTRUCTION OF SILVER DIAMINE FLUORIDE TREATED DENTAL TISSUES

(71) Applicants: University of Kansas, Lawrence, KS (US); University of Southern California, Los Angeles, CA (US)

(72) Inventors: Candan TAMERLER-BEHAR, Lawrence, KS (US); Paulette SPENCER, Lawrence, KS (US); Malcolm L. SNEAD, Los Angeles, CA (US)

(73) Assignees: University of Kansas, Lawrence, KS (US); University of Southern California, Los Angeles, CA (US)

(21) Appl. No.: 18/850,277

(22) PCT Filed: Mar. 24, 2023
(86) PCT No.: PCT/US2023/016316

§ 371 Date: Sep. 24, 2024

Related U.S. Application Data
(60) Provisional application No. 63/323,923, filed on Mar. 25, 2022.

(51) Int. Cl. A61K 8/64; A61Q 11/00; C07K 7/08

(52) U.S. Cl. CPC A61K 8/64; A61Q 11/00; C07K 7/08; C07K 2319/20

(57) ABSTRACT

Described herein are bifunctional peptides, compositions comprising A the same, and methods useful for treatment of dental caries that have been treated with silver diamine fluoride. Treatment with bifunctional peptides remineralizes the carious region to address undesirable tooth discoloration associated with silver diamine fluoride treatment.

### CROSS-REFERENCE TO RELATED APPLICATIONS

This application is a U.S. National Stage Application under 35 U.S.C. § 371 of International Patent Application No. PCT/US2023/016316, filed Mar. 24, 2023, which claims the benefit of and priority to U.S. Provisional Patent Application No. 63/323,923, filed Mar. 25, 2022, the entire contents of each of which are incorporated by reference herein in their entirety for any and all purposes.

### STATEMENT OF U.S. GOVERNMENT SUPPORT

This invention was made with government support under DE025476 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Aug. 10, 2023, is named 104434-0333_SL.txt and is 12,799 bytes in size.

### BACKGROUND

Throughout this disclosure, various publications, patents, and published patent specifications are referenced by an identifying citation. Also within this disclosure are Arabic numerals referring to referenced citations, the full bibliographic details of which are provided subsequent to the Examples section. The disclosures of these publications, patents, and published patent specifications are hereby incorporated by reference into the present disclosure to more fully describe the present technology.

No infectious disease is more common than dental caries (also referred to by the general population as cavities). A factor in its prevalence is the maternal transmission of cariogenic microbiota.^{1, 2} Early childhood caries (ECC) is widely recognized as a global health crisis, with dental caries still the most prevalent chronic disease in children worldwide.³⁻¹² Impacting young children under age five, ECC is characterized by the presence of one or more carious lesions of the primary teeth. ECC has a higher prevalence in children of lower socioeconomic groups with limited access to dental care.^{9,13-15} In the United States, the prevalence of ECC is estimated to be between 3% and 6% of all pre-school aged children, a value which is consistent with literature reviews confirming rates from 1% to 12% in the most developed countries worldwide.^{9,14,16,17} However, the worse health outcomes are disproportionately skewed to certain populations as the risk of ECC in disadvantaged populations and in less developed countries can be as high as 70%.^{9,13,14,18} Once a child develops carious lesions, the disease becomes more difficult and more expensive to control—rapid disease progression is common without immediate professional intervention. As the ECC disease progresses, treatment options diminish and are often very costly. Children with severe early childhood caries must commonly be treated under general anesthesia.^{5,18,19} Due to the high cost and potential comorbidities, general anesthesia may not be an option for all children. More devastating to the development of the dentition and growth of the jaws is that the current standard of care in advanced ECC cases recommends premature extraction or extensive dental restorations. Early extraction of the primary teeth can alter jaw growth, leading to the failure of the remaining and adult replacement teeth to work together effectively during chewing. This can lead to consequential changes in micro- and macro-nutrition that adversely impact child health across a lifetime.^{18,20} Therefore, alternative treatment options that circumvent the cascade of failure described above have been the focus of attention in the dental public health community.

### SUMMARY

Caries is the most ubiquitous infectious disease of mankind and early childhood caries (ECC) is the most prevalent chronic disease in children worldwide, with the resulting destruction of the teeth recognized as a global health crisis. Recent Federal Drug Administration (FDA) approval for the use of silver diamine fluoride (SDF) in dentistry offers a safe, accessible, and inexpensive approach to arrest caries progression in children with ECC. However, discoloration, i.e., black staining, of demineralized or cavitated surfaces treated with SDF has limited its widespread use. Furthermore, SDF treatment reduces effective bonding of adhesive dental composite materials commonly used to mask the staining and restore the function of the carious teeth. Therefore, there is a need for compositions and strategies to mitigate the black staining associated with SDF treatment and remineralize carious regions treated with SDF. The present technology addresses this need.

In an aspect, a bifunctional peptide is provided of amino acid sequence SYEKSHSQAINTDRT-X₁-EQLGVRKELRGV (SEQ ID NO: 1) or one or both of a pharmaceutically acceptable salt thereof and a solvate thereof, where X₁ is absent (a bond) or is a spacer of 1, 2, 3, 4, 5, 6, 7, 8, 9, or 10 amino acids. The bifunctional peptide of the present technology achieves 1) targeted surface binding to silver nanostructures on dental surfaces and 2) the growth of new calcium phosphate mineral layers on the dental surface. The bifunctional peptide can self-assemble on complex surfaces having silver ions or nanostructures thereon to produce an interface serving to modulate functions that favorably direct biomineralization. In any embodiment herein, X₁ may be EAAAK (SEQ ID NO: 2), APA, GGG, PAPAP (SEQ ID NO: 5), or GSGGG (SEQ ID NO: 6).

In an aspect, a composition is provided that includes a bifunctional peptide of the present technology and a pharmaceutically acceptable carrier. The composition may be a powder, syrup, emulsion, suspension, or solution. The bifunctional peptide may be present in the composition in a concentration of about 20 μM to about 150 μM.

In an aspect, a method of treating a dental surface is provided, where the method includes administering silver diamine fluoride (SDF) to the dental surface; and after administering the SDF, administering an effective amount of a bifunctional peptide of the present technology and/or a composition of the present technology to the dental surface. The dental surface may be a dental enamel and/or dentin. The dental enamel may include at least one of a carious region or a hypomineralized region; the dentin may include at least one of a carious region or a hypomineralized region. Administering SDF to the dental surface may include administering an SDF solution to the dental surface to arrest dental caries. The SDF solution may have an SDF concentration of about 38% weight by volume (w/v). Administering the effective amount of the composition may include contacting the dental surface with the composition of the present technology for about 1 minute to about 4 hours. Administering the effective amount of the composition of the present technology may include contacting the dental surface with the composition of the present technology for about 1 minute to about 10 minutes or about 2 hours to about 4 hours. Administering the effective amount of the composition of the present technology may include adding new mineral to the dental surface. Administering the effective amount of the composition of the present technology may reduce visible discoloration on the dental surface resulting from administering the SDF. After administering the effective amount of the composition, the method may include applying an adhesive dental composite to the dental surface.

### BRIEF DESCRIPTION OF THE DRAWINGS

FIG. 1 provides an illustrative schematic of a slab of dental tissue discolored by silver diamine fluoride (SDF).

FIG. 2 depicts a schematic of a bifunctional peptide including a silver-binding domain (AgBP) and a fluorescent domain (DsRed) targeting a dental region treated with SDF, according to the working examples. FIG. 2 discloses SEQ ID NO: 7.

FIG. 3 shows bright field and fluorescence images of an SDF-treated dental slab exposed to DsRed-AgBP, according to the working examples. Scale bars are 1000 mm for the bright field image and 100 μm for the fluorescence image.

FIG. 4A is a bar graph comparing the pairwise mean distance of AgBP domain structures in bifunctional peptides with different spacers, according to the working examples.

FIG. 4B is a schematic showing different views of a single-domain structure in AgBP, according to the working examples.

FIG. 4C is a bar graph comparing the pairwise mean distance of mineralizing peptide (MP) domain structures in bifunctional peptides with different spacers, according to the working examples.

FIG. 4D is a schematic showing different views of a single-domain structure in MP, according to the working examples.

FIG. 5 shows bifunctional peptide structures with different spacers between AgBP and MP, according to the working examples.

FIG. 6A compares optical microscope images of mineral deposition on a silver-coated surface treated with the bifunctional peptide MP-APA-AgBP (bottom) to a control (top), according to the working examples. Scale bars are 830 mm (top) and 195 mm (bottom)

FIG. 6B compares SEM images of mineral deposition on a silver-coated surfaces treated with the bifunctional peptide MP-APA-AgBP (bottom) to a control (top), according to the working examples. Scale bars are 100 μm (top) and 25 μm (bottom).

FIG. 6C shows corresponding energy-dispersive X-ray spectroscopy (EDS) spectra of the SEM images in FIG. 6B, according to the working examples.

FIG. 7 shows mineralization by the MP-APA-AgBP bifunctional peptide on dental tissue treated with SDF, according to the working examples.

FIG. 8 shows SEM images and corresponding EDS spectra from enamel tissue of the dental slab in FIG. 7, with the untreated surface (left), SDF-treated surface (middle), and MP-APA-AgBP bifunctional peptide treated surface (right), according to the working examples.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C; A and B; A and C; or B and C.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

As used herein, the term “peptide” refers to a polymer of amino acid residues joined by amide linkages, which may optionally be chemically modified to achieve desired characteristics. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include unnatural amino acids or residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

By “pharmaceutically acceptable” is meant a material that is not biologically or otherwise undesirable, e.g., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

Pharmaceutically acceptable salts of peptides described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

The peptides of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

As used herein, “subject” refers to an animal, such as a mammal (including a human), that has been or will be the object of treatment, observation or experiment. “Subject” and “patient” may be used interchangeably, unless otherwise indicated. Mammals include, but are not limited to, mice, rodents, rats, simians, humans, farm animals, dogs, cats, sport animals, and pets. The methods described herein may be useful in human therapy and/or veterinary applications. In some embodiments, the subject is a mammal. In some embodiments, the subject is a human.

The term “treatment” or “treating” means administering a compound disclosed herein for the purpose of: (i) delaying the onset of a disease, that is, causing the clinical symptoms of the disease not to develop or delaying the development thereof; (ii) inhibiting the disease, that is, arresting the development of clinical symptoms; (iii) relieving the disease, that is, causing the regression of clinical symptoms or the severity thereof; and/or (iv) alleviating or reducing side-effects of another treatment.

The term “dental surface” refers to a surface of a tooth made of hard tissue that can be treated with the peptides of the present technology. The hard tissue may include enamel, dentin, and/or cementum.

Unless defined otherwise, all technical and scientific terms used herein have the same meaning as commonly understood by one of ordinary skill in the art to which this present technology belongs. Although any methods and materials similar or equivalent to those described herein can also be used in the practice or testing of the present technology, representative illustrative methods and materials are described herein.

### THE PRESENT TECHNOLOGY

There is a continuing need to improve treatment of dental caries. Fluorides have proven useful to slow the progression of dental caries by replacing the hydroxyl group with fluoride in the hydroxyapatite biomineral of the teeth, thereby inhibiting the carious demineralization of teeth.^{3,21-23} Recently, silver diamine fluoride has gained attention as a safe, accessible, and inexpensive approach to arrest caries progression in children with ECC.^{3,4,8,24-26} SDF has been used worldwide for decades but was approved for dental use by the FDA in 2014.^{24,27,28} Although the exact mechanism of action has not been resolved, silver ions deposited on the dental tissues have notable antimicrobial properties.^{29,30} SDF treatment may result in silver ions and silver nanostructures deposited on the dental surface. Recently, silver microwires have been described in teeth treated with SDF, where, without being bound by any theory, the SDF may deposit microwires that replace defects that are a result of caries-provoked demineralization.³¹

Unlike surgical interventions which require skilled professionals, SDF treatments can be applied by a wider range of health care providers.³² SDF works by limiting caries progression and protecting teeth from further degradation.^{3,4,21,24,26,28,33,34} As of 2016, the 38% SDF solution was awarded breakthrough therapy status by the US Food and Drug Administration for use in treating ECC.^{3,4,21,24,26,28,33,34} Numerous case studies have shown the overwhelming benefit of single or bi-annual SDF treatments for caries arrest, with a focus for use of SDF in primary teeth of children affected by ECC.^{3,8,26,28,33,35,36} SDF is inexpensive, application takes minutes, and it does not require significant patient cooperation. Only rare minor gingival irritation and no serious adverse events are associated with the use of SDF.³⁷

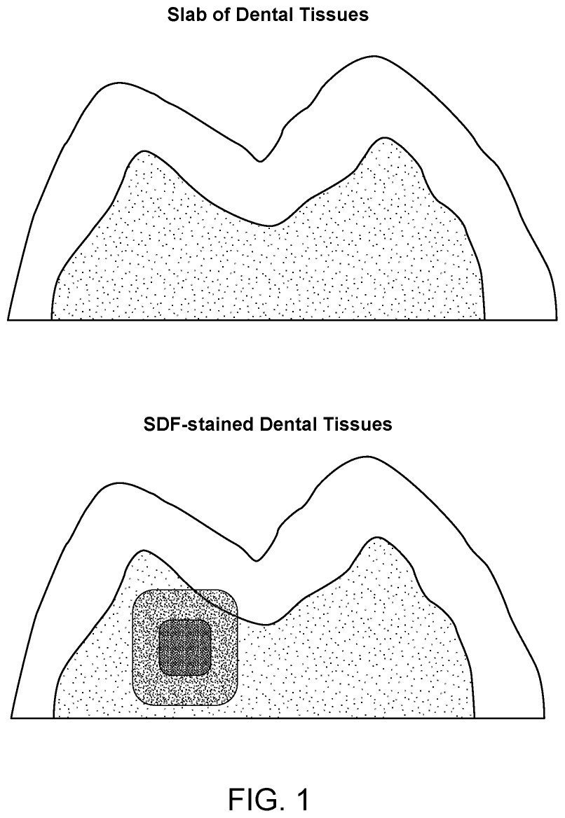

FIG. 1 provides an illustrative schematic of a slab of dental tissue discolored by silver diamine fluoride (SDF). A side effect of SDF is black staining of the treated demineralized or cavitated surfaces due to the deposition of silver metal and ions. This side effect has limited the widespread adoption of SDF treatment for caries arrest. The loss of aesthetics was documented by Crystal et al., who interviewed parents of children qualified to receive SDF treatment and found that roughly one third of parents found the treatment unacceptable under any circumstances.³⁸ In some trials, mild gingiva irritation and redness were noted, but these symptoms subsided in a few days.

SDF is not recommended for use on carious lesions that extend into the dental pulp as it will not arrest the progression of the infection.³⁹ In addition, SDF treatment is recognized to reduce effective bonding of adhesive dental composite materials commonly used to mask the staining and restore the function of the carious teeth.⁴⁰

### Bifunctional Peptides

In an aspect, the present technology provides a bifunctional peptide of amino acid sequence SYEKSHSQAINTDRT-X₁-EQLGVRKELRGV (SEQ ID NO: 1) or one or both of a pharmaceutically acceptable salt thereof and a solvate thereof, where X₁ is absent (a bond) or is a spacer of 1, 2, 3, 4, 5, 6, 7, 8, 9, or 10 amino acids. The bifunctional peptide of the present technology is also alternatively referred to herein as “a peptide of the present technology,” “the peptide of the present technology,” “the peptide,” and the like. The peptide of the present technology may include one or more D-amino acids as well as one or more L-amino acids. In any embodiment herein, the peptide may consist of only D-amino acids, or alternatively in any embodiment herein the peptide may consist only of L-amino acids. As discussed herein, the bifunctional peptide of the present technology consists of a silver binding peptide (AgBP) portion, an optional spacer portion, and a mineralizing peptide (MP) portion. The bifunctional peptide of the present technology achieves 1) targeted surface binding to silver nanostructures on dental surfaces and 2) the growth of new calcium phosphate mineral layers on the dental surface. The bifunctional peptide can self-assemble on complex surfaces having silver ions or nanostructures thereon to produce an interface serving to modulate functions that favorably direct biomineralization.

A bifunctional peptide of the present technology may be synthesized by any technique known to those of skill in the art and by methods as disclosed herein. Methods for synthesizing the disclosed peptides may include chemical synthesis of proteins or peptides, the expression of peptides through standard molecular biological techniques, and/or the isolation of proteins or peptides from natural sources. The disclosed peptides thus synthesized may be subject to further chemical and/or enzymatic modification. Various methods for commercial preparations of peptides and polypeptides are known to those of skill in the art.

A bifunctional peptide of the present technology may alternatively be made by recombinant means or by cleavage from a longer polypeptide. The composition of a peptide may be confirmed by amino acid analysis or sequencing.

The bifunctional peptide includes a functional domain that may target, bind, and/or self-assemble on silver particles, silver nanoparticles, and/or silver surfaces. The silver-binding functional domain in the bifunctional peptides may be used to selectively target tissue treated with SDF. SDF treatment of a dental surface results in silver metal and/or silver ion deposition on the dental surface. The deposited silver from SDF on the dental surface may act as a selective and specific target for the bifunctional peptides binding and/or self-assembly.

The silver-binding functional domain in the bifunctional peptide is a silver binding peptide (AgBP) of amino acid sequence EQLGVRKELRGV (SEQ ID NO: 7). The AgBP may self-assemble on silver particles, silver nanoparticles, and/or silver surfaces, including silver compounds deposited on dental surfaces as a result of SDF treatment to anchor to the SDF-treated surface. A construct with the AgBP domain may have an equilibrium dissociation constant with silver of about 1 to about 3 orders of magnitude lower than a similar construct without the AgBP domain.

In addition to silver-binding, the bifunctional peptides may mineralize and/or remineralize hard tissue. Specifically, the bifunctional peptides include a functional domain to facilitate tissue repair and restoration by depositing calcium phosphate as hydroxyapatite. This functional domain may biomineralize enamel by regulating and initiation the growth of calcium phosphate (e.g., hydroxyapatite) mineral. In this way, the bifunctional peptides direct remineralization of defective enamel and/or dentin.

The MP domain of the bifunctional peptide is of amino acid sequence SYEKSHSQAINTDRT (SEQ ID NO: 8). MP has the ability to control both the kinetics and morphology of calcium phosphate deposition as hydroxyapatite. In this way, MP can facilitate the growth of new mineral layers. As discussed herein, MP has demonstrated capacity to favorably modulate both the kinetics and morphology of calcium phosphate mineral formation. MP modulated mineralization in a manner that closely resembled the function of the amelogenin protein, which exerts a dominant role during enamel biomineralization by hydroxyapatite mineral.⁴⁴ Compared to the amelogenin protein, MP derived from naturally existing amelogenin has increased solubility, greater ease of synthesis, and reduced cost.

A wide range of distinct calcium phosphate phases exist in mineralized tissues and these phases are commonly classified by the Ca/P molar ratio. Biomineralized tissue formed in the presence of a bifunctional peptide of the present technology had an average Ca/P ratio of about 1.20 to about 1.67. Thus, in any embodiment herein, biomineralized tissue formed in the presence of a bifunctional peptide of the present technology may have an average Ca/P ratio of about 1.20, about 1.25, about 1.30, about 1.35, about 1.40, about 1.45, about 1.50, about 1.55, about 1.60, about 1.61, about 1.62, about 1.63, about 1.64, about 1.65, about 1.66, about 1.67, or any range including and/or in between any two of these values. For example, the Ca/P ratio of the biomineralized tissue may have a Ca/P ratio of about 1.35 to about 1.70, including about 1.63 for enamel and about 1.61 for dentine. The biomineralized tissue may have a Ca/P ratio consistent with octacalcium phosphate (having a Ca/P ratio of 1.33), amorphous calcium phosphate (having a Ca/P ratio of 1.50) or hydroxyapatite (having a Ca/P ratio of 1.67), or any value there between.

The bifunctional peptides may or may not have an amino acid sequence between the AgBP and the MP acting as a spacer. The spacer may influence domain activity depending on the spacer's length and flexibility. The spacer sequences may help ensure that the two functional domains MP and AgBP still substantially maintain their isolated functions, and/or may reduce potential interference between the two functional domains AgBP and MP. Such spacer sequences include, but are not limited to, EAAAK (SEQ ID NO: 2), APA, GGG, PAPAP (SEQ ID NO: 5), or GSGGG (SEQ ID NO: 6).

Examples of bifunctional peptides according to the present technology include

(SEQ ID NO: 9)
SYEKSHSQAINTDRTEAAAKEQLGVRKELRGV,
(SEQ ID NO: 10)
SYEKSHSQAINTDRTEQLGVRKELRGV,
(SEQ ID NO: 11)
SYEKSHSQAINTDRTAPAEQLGVRKELRGV,
(SEQ ID NO: 12)
SYEKSHSQAINTDRTGGGEQLGVRKELRGV,
(SEQ ID NO: 13)
SYEKSHSQAINTDRTPAPAPEQLGVRKELRGV,
and
(SEQ ID NO: 14)
SYEKSHSQAINTDRTGSGGGEQLGVRKELRGV.

The bifunctional peptides are engineered peptides that drive nanocomposite formation of calcium phosphate mineral for biomimetic reconstruction of SDF treated tooth tissues. The AgBP domain of the bifunctional peptides targets and binds to SDF-treated dental surfaces. The MP of the bifunctional peptides creates new mineral layers on the SDF-treated dental surface. In this way, the bifunctional peptides remineralize carious dental regions or hypomineralized dental regions that have been treated with SDF. This remineralization may mitigate the black staining resulting from SDF treatment. Additionally, the new mineral formed by remineralizing may act as a stable interface for application of an adhesive dental composite, where the dental composite may be used to fill in holes and cover SDF staining. This aspect of remineralizing is beneficial since a dental composite does not typically adhere well to SDF-treated dental surfaces.

The resulting bifunctional peptides demonstrated both remineralizing and silver-binding functions. The bifunctional peptide binds to silver deposits while also rebuilding damaged dental tissues, adding a new layer of protective calcium phosphate biomimetic mineral nanocomposite. The calcium phosphate nanocomposite formed by the bifunctional peptide may also inhibit caries progression, protect against future caries formation, and/or rebuild damaged dental tissue.

### Compositions

In an aspect, a composition is provided that includes a bifunctional peptide of any embodiment disclosed herein, a pharmaceutically acceptable carrier or one or more excipients, fillers or agents (collectively referred to hereafter as “pharmaceutically acceptable carrier” unless otherwise indicated and/or specified). In a related aspect, a medicament for treating dental caries or hypomineralized dental surfaces is provided that includes a bifunctional peptide of any embodiment disclosed herein and optionally a pharmaceutically acceptable carrier. In a related aspect, a medicament for controlling biomineralization on a dental surface is provided that includes a bifunctional peptide of any embodiment disclosed herein and optionally a pharmaceutically acceptable carrier. In a related aspect, a pharmaceutical composition is provided that includes an effective amount of a bifunctional peptide of any embodiment disclosed herein as well as a pharmaceutically acceptable carrier. For ease of reference, the compositions, medicaments, and pharmaceutical compositions of the present technology may collectively be referred to herein as “compositions.” In further related aspects, the present technology provides methods and uses that include a bifunctional peptide of any aspect or embodiment disclosed herein and/or a composition of any embodiment disclosed herein as well as uses thereof.

“Effective amount” refers to the amount of a compound, bifunctional peptide, or composition required to produce a desired effect. One example of an effective amount includes amounts or dosages that yield acceptable toxicity and bioavailability levels for therapeutic (pharmaceutical) use including, but not limited to facilitating the formation of new calcium phosphate mineral layers on dental surfaces, masking and/or reducing the staining caused by SDF, improving adhesion between a dental adhesive composite and an SDF-treated dental surface, inhibiting caries progression, protecting against future caries formation, and/or rebuilding damaged dental tissue. In any aspect or embodiment disclosed herein (collectively referred to herein as “any embodiment herein,” “any embodiment disclosed herein,” or the like) of the compositions, pharmaceutical compositions, and methods including a bifunctional peptide of the present technology, the effective amount may be an amount effective in treatment, including facilitating the formation of new calcium phosphate mineral layers on dental surfaces, masking and/or reducing the staining caused by SDF, improving adhesion between a dental adhesive composite and an SDF-treated dental surface, inhibiting caries progression, protecting against future caries formation, and/or rebuilding damaged dental tissue. By way of example, the effective amount of any embodiment herein including a bifunctional peptide of the present technology may be from about 0.01 g to about 200 mg of the bifunctional peptide (such as from about 0.1 g to about 50 mg of the bifunctional peptide or about 10 g to about 20 mg of the peptide). The effective amount may be related to the corresponding area and the molecular mass of the bifunctional peptide required to saturate a SDF-treated dental surface. The molecular mass required to deliver the corresponding surface coverage could be obtained by converting the number of bifunctional peptides that is calculated from the theoretical “footprint” for each bifunctional peptide using the variety of peptide structural analyses tools including UCSF Chimera tool. See E. Cate Wisdom, Yan Zhou, Casey Chen, Candan Tamerler, and Malcolm L. Snead, Mitigation of Peri-implantitis by Rational Design of Bifunctional Peptides with Antimicrobial Properties, ACS Biomaterials Science & Engineering 2020 6 (5), 2682-2695 DOI: 10.1021/acsbiomaterials.9b01213. The “theoretical footprint” of the bifunctional peptides could be determined through the length and width distance values measured from the α-carbon of amino acid residues. The number of bifunctional peptides could be next converted to a molecular mass required to deliver the corresponding dental surface coverage. The methods and uses according to the present technology may include an effective amount of a bifunctional peptide of any embodiment disclosed herein. In any aspect or embodiment disclosed herein, the effective amount may be determined in relation to a subject and/or in relation to dental caries. The term “subject” and “patient” may be used interchangeably.

Thus, the instant present technology provides pharmaceutical compositions and medicaments including a bifunctional peptide of any embodiment disclosed herein (or a composition of any embodiment disclosed herein) and a pharmaceutically acceptable carrier. The compositions may be used in the methods and treatments described herein. The pharmaceutical composition may be packaged in unit dosage form. The unit dosage form is effective in treatment, including facilitating the formation of new calcium phosphate mineral layers on dental surfaces, masking or reducing the staining caused by SDF, improving adhesion between a dental adhesive composite and an SDF-treated dental surface, inhibiting caries progression, protecting against future caries formation, and/or rebuilding damaged dental tissue. Generally, a unit dosage including a bifunctional peptide of the present technology will vary depending on patient considerations. Such considerations include, for example, age, protocol, condition, sex, extent of disease, contraindications, concomitant therapies and the like. Further, a unit dosage including a bifunctional peptide of the present technology may vary depending on the size of the carious region and SDF treatment. An exemplary unit dosage based on these considerations may also be adjusted or modified by a physician skilled in the art. Suitable unit dosage forms, include, but are not limited to oral solutions, powders, lozenges, topical varnishes, lipid complexes, liquids, etc.

The pharmaceutical compositions and medicaments may be prepared by mixing a bifunctional peptide of the present technology with one or more pharmaceutically acceptable carriers, excipients, binders, diluents or the like. Such compositions may be in the form of, for example, powders, syrup, emulsions, suspensions, or solutions. The instant compositions may be formulated for various routes of administration, for example, by intraoral administration or via administration (e.g., application) to a dental surface external to a patient. The following dosage forms are given by way of example and should not be construed as limiting the instant present technology.

A bifunctional peptide of the present technology may be incorporated into adhesive formulations as part of a peptide-polymer hybrid and/or impregnated into the adhesive. The adhesive may be applied to the exposed tooth surface. The composite, which is placed on top of the adhesive, forms a bond with the adhesive. Peptide-polymer hybrids may be obtained through tethering to a monomer such as methacrylic acid (MA) and form peptide-monomer pairs. Peptide-monomer pairs can be copolymerized as an integral part of an adhesive. For example, a bifunctional peptide may be converted to monomer tether through a reaction between the amino group in a linker's lysine (K) side chain (N-terminal K) and the carboxylic group in a methacrylate linkage, where the monomer's reactive C═C bond is reserved for subsequent participation in the polymerization reaction. See Sheng-Xue Xie, Linyong Song, Esra Yuca, Kyle Boone, Rizacan Sarikaya, Sarah Kay VanOosten, Anil Misra, Qiang Ye, Paulette Spencer, and Candan Tamerler ACS Applied Polymer Materials 2020 2 (3), 1134-1144 DOI: 10.1021/acsapm.9b00921; Yuca, E.; Xie, S.-X.; Song, L.; Boone, K.; Kamathewatta, N.; Woolfolk, S. K.; Elrod, P.; Spencer, P.; Tamerler, C. Reconfigurable Dual Peptide Tethered Polymer System Offers a Synergistic Solution for Next Generation Dental Adhesives. Int. J. Mol. Sci. 2021, 22, 6552. https://doi.org/10.3390/ijms22126552.

For intraoral administration, powders and suspensions are acceptable as solid dosage forms. These may be prepared, for example, by mixing a bifunctional peptide of the instant present technology with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms may contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents and/or perfuming agents.

Liquid dosage forms for oral administration (e.g., intraoral administration) may be in the form of pharmaceutically acceptable emulsions, syrups, suspensions, or solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral administration.

As noted above, suspensions may include oils. Such oils include, but are not limited to, peanut oil, sesame oil, cottonseed oil, corn oil, and olive oil. Suspension preparation may also contain esters of fatty acids such as ethyl oleate, isopropyl myristate, fatty acid glycerides, and acetylated fatty acid glycerides. Suspension formulations may include alcohols, such as, but not limited to, ethanol, isopropyl alcohol, hexadecyl alcohol, glycerol, and propylene glycol. Ethers, such as but not limited to, poly(ethyleneglycol), petroleum hydrocarbons such as mineral oil and petrolatum; and water may also be used in suspension formulations.

The pharmaceutical compositions and medicaments in liquid or gel form may have a concentration of a bifunctional peptide of the present technology sufficient to provide an effective amount as described above. The concentration of the bifunctional peptide of the present technology in the pharmaceutical compositions and medicaments may be about 5 μM to about 500 μM (including about 5 μM, about 10 μM, about 50 μM, about 100 μM, about 200 μM, about 500 μM, or any range including and/or in between any two of these values).

The pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. The formulations may optionally contain stabilizers, antimicrobial agents, antioxidants, pH modifiers, surfactants, bioavailability modifiers, and combinations of these. The carriers and stabilizers vary with the requirements of the particular composition, but typically include nonionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Powders and sprays may be prepared, for example, with excipients such as lactose, talc, silicic acid, aluminum hydroxide, calcium silicates and polyamide powder, or mixtures of these substances. Ointments, pastes, creams, and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), and “Remington: The Science and Practice of Pharmacy,” 20^{th} Edition, Editor: Alfonso R Gennaro, Lippincott, Williams & Wilkins, Baltimore (2000), each of which is incorporated herein by reference.

### Methods

Disclosed herein, in one aspect, is an method for facilitating the formation of new calcium phosphate mineral layers on dental surfaces treated with SDF, thereby masking or reducing the staining caused by SDF and improving adhesion between a dental adhesive composite and an SDF-treated dental surface by applying a bifunctional peptide of the present technology to a dental surface, e.g., to produce a film on the dental surface.

In another aspect, provided herein are methods of treating dental caries in a subject in need thereof, the methods comprising, consisting essentially of, or consisting of administering an effective amount of a bifunctional peptide of the present technology or a composition of the present technology to a dental surface treated with SDF in the subject.

In any of the methods disclosed herein, an effective amount of bifunctional peptide or a composition containing the bifunctional peptide may be administered to a dental surface for a period of about 10 seconds to about 4 hours, such as for a period of about 10 seconds to about 20 seconds, about 20 seconds to about 1 minute, about 1 minute to about 4 hours, 1 minute to 10 minutes, or 2 hours to 4 hours.

Treatment with the bifunctional peptide may be added to the FDA-approved SDF treatment regime to further increase the arrest of caries progression, mediate remineralization at the SDF-treated dental surface, and mitigate SDF's adverse effect, i.e., black staining of the treated carious lesion.

The bifunctional peptides may work synergistically with the SDF treatment to help protect and rebuild damaged dental tissues while adding a new mineral layer that may be incorporated in adhesive dental composites to restore function and esthetics to people suffering from dental caries. As discussed previously herein, a bifunctional peptide of the present technology may be incorporated in an effective amount into adhesive formulations as part of a peptide-polymer hybrid and/or impregnated into the adhesive. The adhesive is applied to the exposed tooth surface. The composite, which is placed on top of the adhesive, forms a bond with the adhesive.

Few studies have been published to date focusing on masking or reducing the staining of SDF on carious dentin and enamel and have used potassium iodide, composites, or glass ionomer cement.⁸³⁻⁸⁵ Hamdy et. al. used a calibrated spectrophotometer to monitor changes in tooth color following SDF treatment and found a composite coating was the most successful masking agent at baseline and after an aging protocol of the three treatments.⁸⁴ However, the composite treatment that immediately followed SDF application used an etching step and multiple coats of dental composite on the affected surface and the effectiveness of the initial SDF treatment after the composite coverage was not reported. Further, it is known that composite restorations in the oral cavity have a high rate of failure due to cracks and gaps creating spaces for bacterial infiltration causing further decay.⁸⁶⁻⁸⁹

A bifunctional peptide of the present technology may provide a new mineral layer on the SDF-treated dental surface to improve adhesion between a dental adhesive composite and the SDF-treated dental surface. The bifunctional peptides of the present technology, having AgBP domains for targeting silver, help reinforce interfacial binding between silver on the dental surface and the composite. In this way, the bifunctional peptides improve interfacial integrity and binding of polymeric composites to dental surfaces.

In another aspect, the bifunctional peptide may be mixed with a dental adhesive composite prior to application to the dental surface to improve adhesion between the composite and the SDF-treated dental surface.

Another approach to mask or reduce the staining caused by SDF is to remineralize the affected dentin and/or enamel using a bifunctional peptide of the present technology. Bacteria associated with dental caries works to break down the tooth structure and the fluoride component of SDF treatment promotes natural remineralization of a treated tooth.^{25,81,91} Furthermore, the bifunctional peptides of the present technology are capable of directing remineralization of calcium phosphate materials.

The examples herein are provided to illustrate advantages of the present technology and to further assist a person of ordinary skill in the art with preparing or using the bifunctional peptides and compositions of the present technology. The examples herein are also presented in order to more fully illustrate the preferred aspects of the present technology. The examples should in no way be construed as limiting the scope of the present technology, as defined by the appended claims. The examples may include or incorporate any of the variations, aspects, or embodiments of the present technology described above. The variations, aspects, or embodiments described above may also further each include or incorporate the variations of any or all other variations, aspects, or embodiments of the present technology.

### Examples

### Materials and Methods

### Bifunctional Peptide Design

The AgBP (EQLGVRKELRGV) (SEQ ID NO: 7) 12 amino acid sequence was selected for use in the bifunctional peptide. Binding confirmation studies were completed using DsRed-AgBP to visualize binding to the SDF-treated tooth samples by the fluorescent reporter, DsRed protein. Production of DsRed-labeled solid binding peptides has been reported in previous publications.^{61,62} For the bioactive remineralization portion of this work, a 15 amino acid sequence MP, SYEKSHSQAINTDRT (SEQ ID NO: 8), was selected.

The studies herein designed a bifunctional peptide including MP and AgBP peptide sequences. The two peptide sequences were joined by a short, flexible spacer designed using biocomputational modeling integrated with experimental studies to ensure individual activities were maintained in the bifunctional peptide. The pairwise displacement of alpha carbons of the respective peptide domains were analyzed among a selection of candidate spacer sequences. PEP-FOLD3 was used to generate candidate chimeric peptide structures.⁶³ The structures were compared pairwise for each structure between groups for the sum of squares of distances between corresponding alpha carbons, calculated by RRDistMaps.⁶⁴ These comparisons were normalized by the internal mean distance for the domain-only structures. The mean distance difference was expressed as the percentage increase over the self-group mean distances of the individual domains. The spacer sequence with a smaller value for the sum of this difference was selected for the bifunctional peptide because it represented a smaller change in computationally predicted average alpha carbon distance.

### Peptide Design

The bifunctional peptide was synthesized using standard Fmoc solid-phase peptide synthesis on the AAPPTec Focus XC (AAPPTec, KY, USA) automated peptide synthesizer, following previous published protocols.^{50,51,65} Following synthesis, the peptide was cleaved from the resin and side-chain-deprotected using a cleavage cocktail (trifluoroacetic acid/phenol/ethanedithiol/triisopropylsilane/DI water (87.5:5:2.5:2.5:2.5) for 2 hours and precipitated in cold ether. Crude peptide purification was performed by semi-preparative reverse-phase high performance liquid chromatography (HPLC) on a Waters system, composed of a Waters 600 controller and a Waters 2487 Dual Absorbance Detector, using a 10 μm C-18 silica Luna column (250×10 mm, Phenomenex Inc., CA, USA). The mobile phase was 94.5% HPLC-grade water, 5% chromplete acetonitrile, and 0.1% trifluoroacetic acid (phase A), and 99.9% chromplete acetonitrile and 0.1% trifluoroacetic acid (phase B). Lyophilized peptide was dissolved in 3 mL of phase A, then purification was executed in a 0.5% phase B·min⁻¹ linear gradient from 5% B to 85% B, with a 3 mL·min⁻¹ flow rate at room temperature and detection at 254 nm. Fractions collected were verified via analytical HPLC on a Shimadzu system, composed of a Shimadzu LC-2010 HT liquid chromatograph and an SPD-M20A prominence diode array detector, using a 5 μm C-18 silica Luna column (250×4.6 mm, Phenomenex Inc., CA, USA). The mobile phase was 99.9% HPLC-grade water and 0.1% trifluoroacetic acid (phase A), and 100% acetonitrile (phase B). The system operated on a linear gradient under these conditions: 1 mL·min⁻¹ flow rate; detection at 254 nm; 10 μL sampling loop; 40° C. temperature. The purified bifunctional peptides were then lyophilized and stored at −20° C.

### Peptide Functionalization of Dental Tissue

Lyophilized peptide stocks were selected and reconstituted in Milli-Q (resistance, >18 MQ) water immediately prior to experiments with both silver-coated silica substrates and slabs of dental tissue. Stock solutions were prepared to a final concentration of 100 μM peptide. Silica substrates or slabs of dental tissue were submerged into a solution containing 50 μM peptide and allowed 2-4 hours gentle shaking at room temperature to ensure the binding of AgBP. This excess concentration and incubation time was used to ensure ample peptide binding.

### Silver-Coated Silica Substrates

To confirm the selectivity and specificity for silver-binding of bifunctional peptides, preliminary studies were conducted on silver-coated silica substrates. Silica wafers were cut into 1 cm square pieces using a diamond scribe and a published protocol for electrolysis deposition of silver onto silica was followed.^{66,67} Briefly, the Si(111) wafers were cleansed in hydrogen peroxide and sulfuric acid solution (3:7) for 20 minutes, rinsed thoroughly with Milli-Q water, and dried with N₂ gas prior to use. An etching step was employed prior to Ag-deposition, where a drop of 5% NH₄F solution was placed on the surface of the cleaned silica substrate for 30 s and removed carefully with N₂ gas. A plating solution was prepared with 0.020 M NH₄F and 0.010 M AgNO₃ and placed onto the pre-etched silica substrates for about 60 s to allow for silver deposition. After the reaction occurred, the substrates were washed with Milli-Q water.

### Preparation of Slabs of Dental Tissues

Specimens of dental tissues were prepared using a protocol modified slightly from previous publications.⁴⁵ The specimens consisted of extracted human molars. These teeth would otherwise be discarded, no patient identifiers are associated with the teeth, and thus, this is not considered human subject research. The extracted teeth were stored at 4° C. in 0.9% wt./vol. NaCl containing 0.002% sodium azide. Teeth exhibiting carious lesions were selected and the root structure was removed by sectioning perpendicular to the long axis of the molar using a water-cooled low-speed diamond-blade saw (Buehler, Lake Bluff, IL). The tooth was sectioned parallel to the long axis to provide 1.4 mm thick slabs. The prepared sections were stored in a modified phosphate buffered saline (PBS) buffer (20 mM Na₂HPO₄, 137 mM NaCl, 2.7 mM KCl, and 1.8 mM KH₂PO₄, pH 7.4) at 4° C. until experimental use.

### SDF Treatment Protocol

SDF was applied to dental tissue following the detailed guidelines published by the UCSF Caries Arrest Committee.^{25,34} Briefly, the prepared slabs of dental tissue were removed from the modified PBS buffer, rinsed with Milli-Q purified water, and gently dried with compressed air to reveal suitable carious regions of the dental tissues. A single drop of commercially available 38% SDF solution (Dengen Caries Arrest; Dengen Dental, Bahadurgarh, Haryana, India) was placed into a fresh plastic dish. A microbrush applicator head was dipped into the solution and touched to the side of the dish to remove excess fluid by surface tension. The SDF was applied to the dental tissue, allowed to absorb for 1 minute and the treated sample was gently rinsed with Milli-Q purified water. The tissue was placed back into modified PBS buffer and incubated at 37° C. for 6 hours to mimic the oral environment and to allow for SDF activity. The progressive darkening of the treated regions due to precipitation of silver and fluoride from the SDF on the dental tissue was noted.

### Enzyme-Mediated Biomineralization

To investigate calcium phosphate nucleation facilitated by the MP, an alkaline phosphatase (AP) based mineralization model was followed.⁴⁴ This model mimics the biological process wherein inorganic phosphate is cleaved from organic phosphate by naturally occurring AP and subsequently reacts with free calcium. Briefly, control samples without peptide and test samples functionalized with MP were submerged in a remineralization buffer containing 14.4 mM β-glycerophosphate (β-GP) and 24 mM Ca²⁺ in 25 mM Tris-HCl at pH 7.4. Mineralization reactions were initiated by adding alkaline phosphatase (fastAP; ThermoFisher, USA) to the solutions to achieve a final concentration of 1.4×10⁻⁶ g/mL and allowed to react at 37° C. for 2 hours to form a calcium phosphate mineral layer on the sample surface. Samples were removed from the solution and allowed to air dry prior to microscopic and spectroscopic analyses.

### Fluorescence and Optical Microscopy

Optical microscopic images of dental tissue and Ag-coated silica substrates were taken at 10× magnification on a Nikon SMZ800 StereoMicroscope (Nikon Instruments Inc., NY, USA) system equipped with a Q-Imaging MicroPublisher 5.0 RTV camera (Teledyne Qimaging, Teledyne Photometrics, AZ, USA). Images were processed with the Q-Capture software (Teledyne Photometrics, AZ, USA). Fluorescence images were acquired using a Leica TCS SPE Laser Scanning Confocal DM6-Q upright microscope (Leica Microsystems, NC, USA) equipped with a 10× Leica Infinity corrected objective. DsRed fluorescence was captured using a laser excitation of 561 nm. Images were processed using the Leica LAS-X imaging software.

### Mineral Characterization

To image the mineral morphology, surface coverage, and to determine the Ca/P ratios of the precipitated material, a Hitachi SU8230 Field Emission Scanning Electron Microscope (Hitachi High-Tech America, IL, USA) equipped with a silicon drift EDS detector (X-Max, Oxford Instruments, MA, USA) was used. SEM imaging was completed at 5 kV and EDS measurements made with an acceleration voltage of 10 kV to improve signal. Prior to SEM/EDS analysis, samples were sputter-coated with 5 nm iridium using a Quorum sputter coating system (Q150, Quorum, UK). EDS analysis was performed using AZtec software (Oxford Instruments, MA, USA) and values were averaged across a minimum of three regions for each sample.

### Results

Silver Binding Peptide Assembles onto SDF-Treated Dental Tissues

Restoring diseased dental tissue has multiple challenges. The silver binding peptide AgBP had specific and selective binding attributes to anchor to silver nanoparticles and surfaces. The AgBP was investigated for its specific targeting and binding properties onto the SDF treated carious dental tissues.

Fluorescence fusion proteins with a peptide tag that self-assembles on metallic nanomaterials may be used as bimodal imaging nanoprobes.^{53,54,61,62} Engineered fusion green- or red-fluorescent proteins (GFP and DsRed, respectively) were developed by incorporating silver binding (AgBP) and gold binding peptides (AuBP). Fluorescent labelling using small fluorophores commonly requires additional labeling steps, whereas the fluorescence proteins with the metal binding peptide tags as a fusion partner offer to identify the location of the peptide in a single step to monitor surface modification and the self-assembly process.

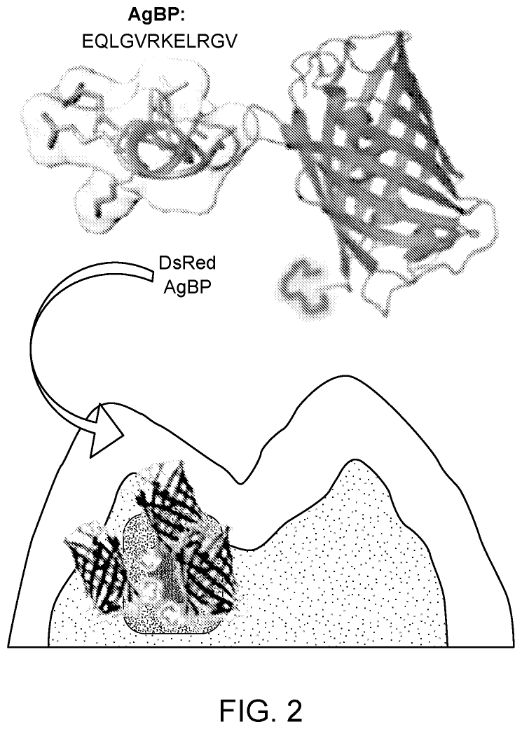

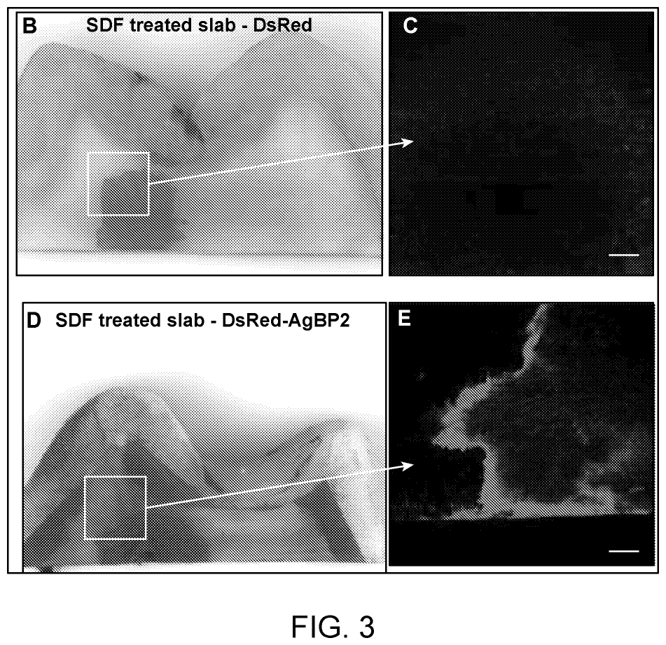

Here, a DsRed-AgBP fusion protein was employed for direct visualization of silver binding ability of AgBP via the DsRed fluorescence reporter protein onto the SDF-treated slabs of dental tissue shown in FIGS. 2 and 3. The DsRed protein without a AgBP tag was used as the control on the SDF-treated dental tissue and this resulted in only minor fluorescent, whereas a robust red fluorescent signal was observed on the SDF treated dental tissue functionalized by DsRed-AgBP, as shown in FIG. 3. The result confirmed that the selective silver binding property of the AgBP was preserved in the presence of the fluorescent signal showing colocalizing only to the silver treated areas following silver diamine treatment of the dental tissue.

### Bifunctional Peptide Design Targeting SDF Treated Dental Tissues

Building upon the observed AgBP binding to silver deposits of the SDF-treated dental tissue, a bifunctional peptide was engineering that contained an AgBP domain and a peptide domain driving mineral formation. For this mineral formation function, a mineralizing peptide (MP) having an amino acid sequence SYEKSHSQAINTDRT (SEQ ID NO: 8) was selected. The MP imparts fast remineralization kinetics as well as control of crystallite morphology in a manner similar to naturally derived MP amelogenin protein, the most abundant protein associated with enamel formation in mammalian teeth.⁴⁴⁵⁹ The MP regulated formation of a robust hydroxyapatite mineral layer on demineralized, human dentin root surfaces in a cell-free assay.

When combining different peptide functionality, i.e., mineralization and metal binding, a challenge to address lies in the design of a spacer between the two peptide domains that reduces potential interference between the domains. Therefore, several spacers of various amino acid sequence were investigated as the linking element between the MP and AgBP domains. Spacers influence domain activity through length and flexibility.

### Computational Analysis of Peptide Design

An “in-solution” structure prediction⁶³ approach was sued to estimate the changed folding dynamics of the bifunctional peptide compared to the single-domain peptides.⁶⁴ The structural variations examined included changes to diffusion rates between the bifunctional domain attributes and the changes to backbone conformation for each domain of the bifunctional peptide depending on the relative orientations of the domains. Since the diffusion rate for longer peptides generally decreases, bifunctional peptides may diffuse more slowly than their single domain counterparts. While slower diffusion of bifunctional peptides may limit some applications, for systems in which the material surface-peptide interaction offers clear competitive advantages when surface assembly occurs, the slower diffusion is outweighed. This limitation may be overcome for systems in which the material surface-peptide interaction offers competitive advantage for surface assembly. Also, the change in the backbone conformation may influence the free energy of adsorption and desorption when comparing single-domain and bifunctional peptides. The objective for spacer design was to reduce the backbone changes in computationally folded structures. Bifunctional peptide structures were compared to single domain structures in order to select the spacer sequence which reduced the structural change, according to the following formula,

$$minimize : \sum ( min {\sum }_{single , chimeric}^{Paired Structures between Clusters} {\vert {C}_{α , single} ( x , y , z ) - {C}_{α , chimeric} ( x , y , z ) \vert}^{2} )$$

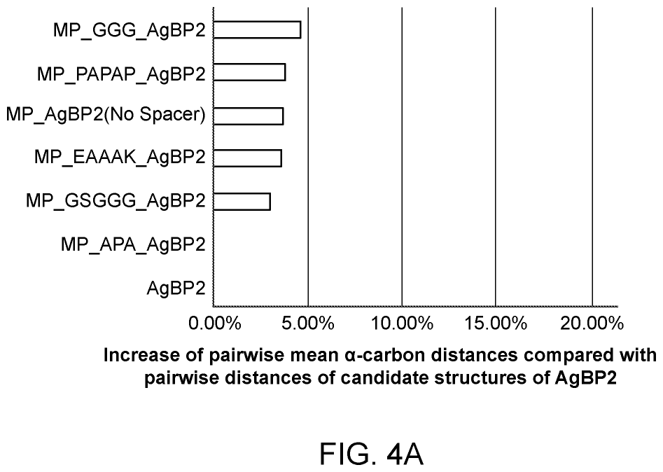

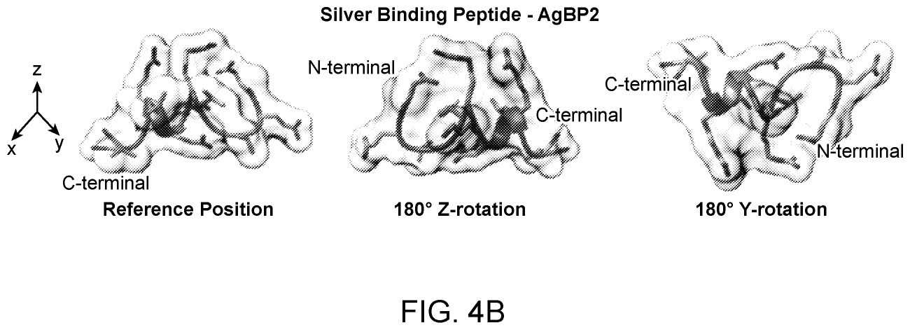

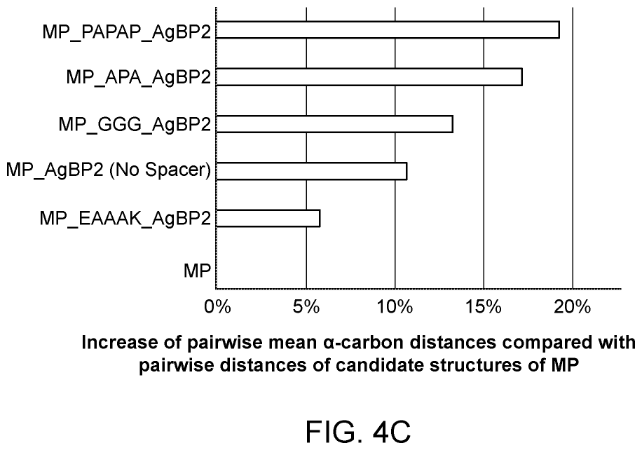

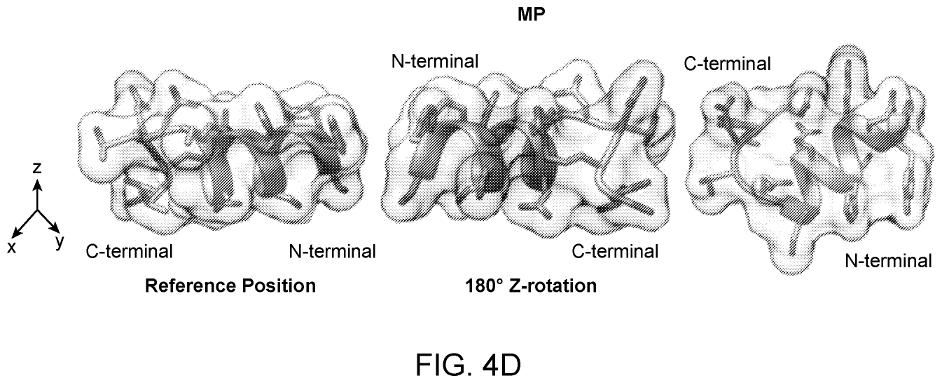

The backbone alpha-carbons in each peptide domain were compared between structures. Specifically, the overlapping sequence parts between the bifunctional peptide sequence and the single domain were compared. Without being bound by any theory, larger distances between alpha-carbons may indicate larger folding changes for the domain, with the implication that the probability of domain function change was proportional to the folding change. The distances were likely spacer-dependent. FIGS. 4A to 4D shows that selected spacers result in different proximities and different orientations between the domains AgBP and MP. The proximities and orientation between the domains strongly influenced the observed peptide backbone shape changes for each individual domain. FIG. 4A shows that the AgBP domain had a smaller change in backbone folding than the MP domain shown in FIG. 4C.

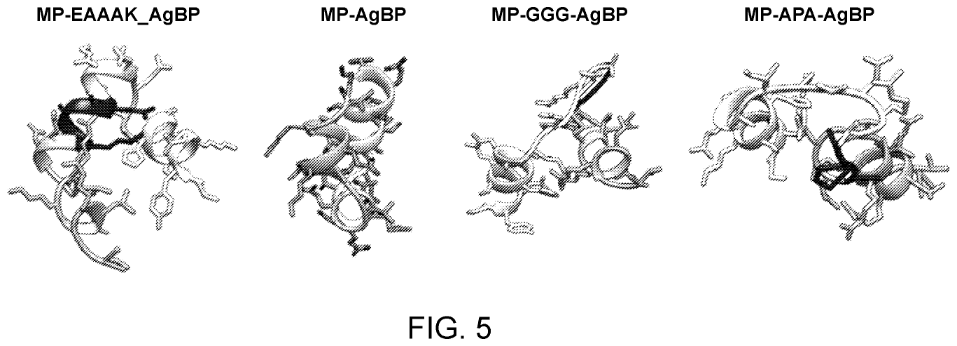

The distances between the single domain (2 cluster representatives for AgBP and 5 cluster representatives for MP) compared to the bifunctional peptides (5 cluster representatives for each sequence) was characterized by the mean sum of squares of distances between corresponding alpha carbons by cluster, as shown in FIGS. 4A to 4D. These mean sums of squares were scaled by the single domain comparison. The self-comparison is the mean of the pairwise distances between different cluster representatives. When these percentages were summed for both single domains for each chimeric sequence, spacer EAAAK (SEQ ID NO: 2) resulted in the smaller predicted backbone change (9.40) followed by APA (17%) and the GGG spacer (180%). Using no spacer in the chimeric sequence resulted in a summed percentage of 1400. The alpha-helical spacer of EAAAK (SEQ ID NO: 2) results in the smaller predicted change in backbone conformation among spacers evaluated. Table 1 gives the summed percentages for candidate bifunctional peptide sequences. FIG. 5 shows 3D models of computationally folded structures for the four bifunctional peptides with the smaller changes in their backbone.

TABLE 1
Distance between AgBP and MP and backbone change
in the bifunctional peptides compared to the
single domain residues.

| | | Summed |
|---|---|---|
| | | Backbone |
| | | Change |
| Name | Sequence | (%) |
| MP | SYEKSHSQAINTDRT | N/A |
| | (SEQ ID NO: 8) | |
| AgBP | EQLGVRKELRGV | N/A |
| | (SEQ ID NO: 7) | |
| MP_EAAAK_ | SYEKSHSQAINTDRTEAAAKEQLGVRKELR | 9.4 |
| AgBP | GV (SEQ ID NO: 9) | |
| MP_AgBP | SYEKSHSQAINTDRTEQLGVRKELRGV | 14 |
| (No | (SEQ ID NO: 10) | |
| Spacer) | | |
| MP_APA_ | SYEKSHSQAINTDRTAPAEQLGVRKELRGV | 17 |
| AgBP | (SEQ ID NO: 11) | |
| MP_GGG_ | SYEKSHSQAINTDRTGGGEQLGVRKELRGV | 18 |
| AgBP | (SEQ ID NO: 12) | |
| MP_PAPAP_ | SYEKSHSQAINTDRTPAPAPEQLGVRKELR | 23 |
| AgBP | GV (SEQ ID NO: 13) | |
| MP_GSGGG_ | SYEKSHSQAINTDRTGSGGGEQLGVRKELR | 25 |
| AgBP | GV (SEQ ID NO: 14) | |

### Functional Activity of MP-APA-AgBP on Ag-Coated Silica Substrates

The MP peptide was selected for use in these studies based on its observed rapid kinetics for calcium phosphate precipitation and favorable mineral crystallite morphology. The spacer APA was selected for use based on its likelihood to maintain functionality of each domain, as described above. The preservation of activity contributed from each functional domains in the bifunctional peptide having an APA spacer was investigated on Ag-coated silica.

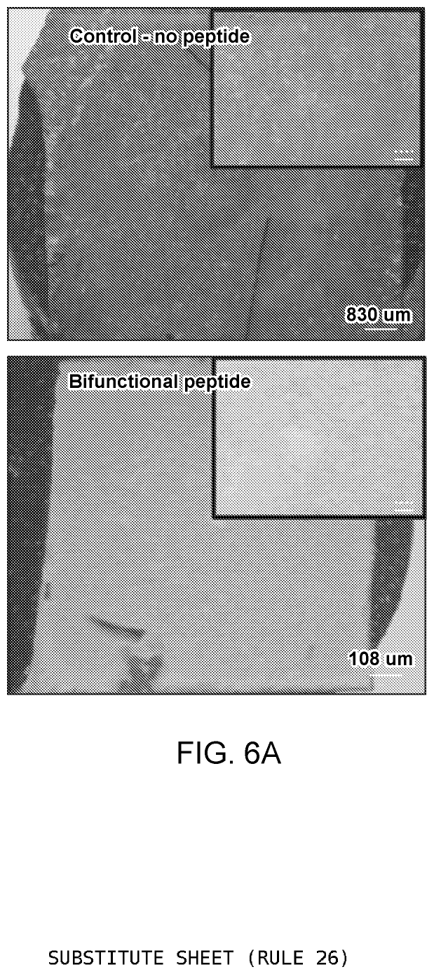

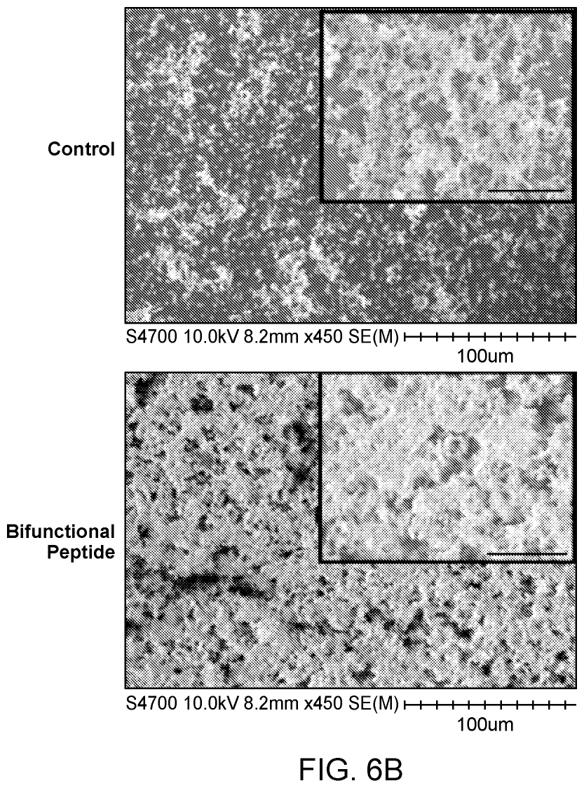

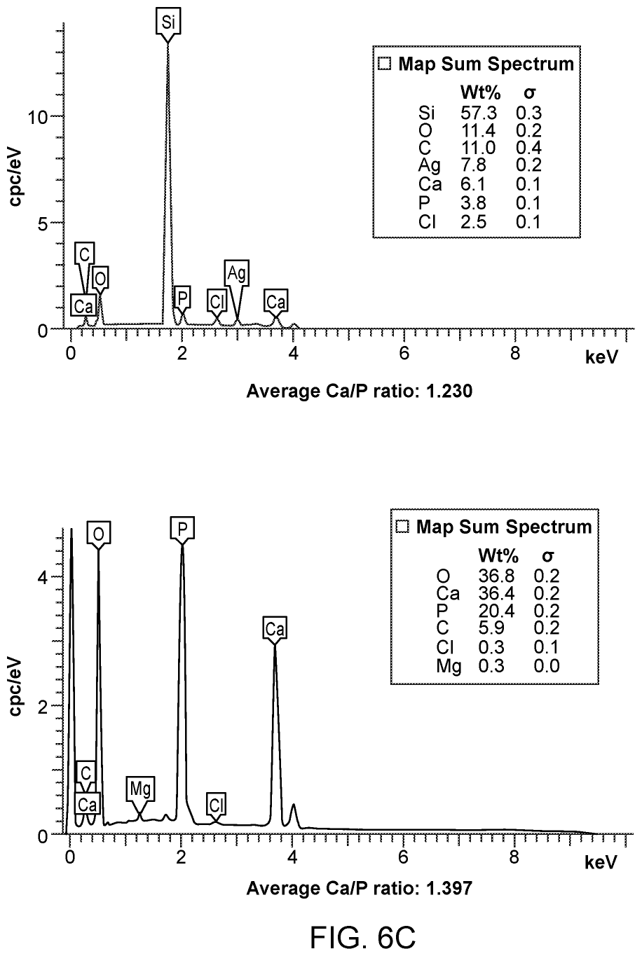

FIG. 6A shows representative optical images of the Ag-coated substrates with no peptide (control, top) or with 50 μM bifunctional peptide (experiment, bottom). The samples were subjected to an enzyme-mediated mineralization protocol, mimicking biomineralization processes in nature. The samples were gently washed with water and the formed mineral layer was dried overnight prior to SEM/EDS analysis. Alkaline phosphatase-based mineralization resulted in significantly higher mineral coverage in the presence of the bifunctional peptide compared to control samples, as shown in FIG. 6B. FIG. 6C shows EDS results that confirmed the formation of a calcium phosphate mineral isomorph from which the calcium/phosphate (Ca/P) ratios were calculated to compare potential mineral compositional differences among samples. The mineral layer formed in the presence of the bifunctional peptide had an average Ca/P ratio of about 1.40 as compared to 1.23 for control samples, as shown in FIG. 6C.

A wide range of distinct calcium phosphate phases exist in mineralized tissues and these phases are commonly classified by the Ca/P molar ratio.⁷⁷⁻⁸⁰ The molar ratio of 1.40 falls between that of octacalcium phosphate and amorphous calcium phosphate with Ca/P ratios of 1.33 and 1.50, respectively. The results confirm that the bifunctional peptide preserved its mineralization activity.

### Bifunctional Peptide Enabled Mineralization on SDF-Treated Dental Tissues

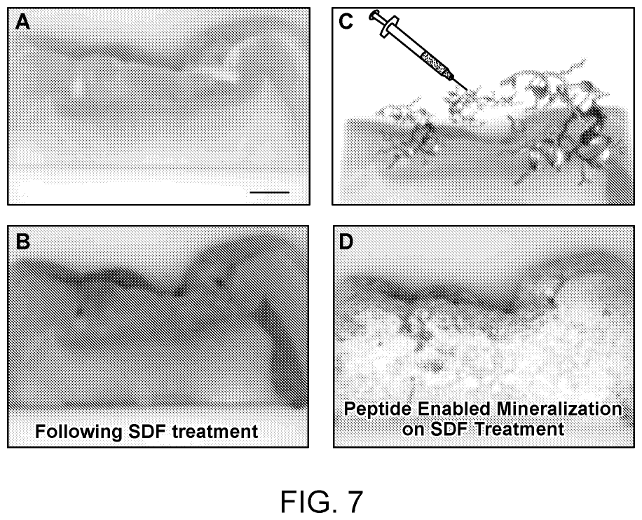

Building upon the success of the bifunctional peptide mineralization on silver coated surfaces, four SDF treated slabs of carious dental tissue were prepared. The bifunctional peptide with the APA spacer demonstrated the capacity to direct mineralization onto the SDF treated dental tissues. FIG. 7 reveals the new mineral coverage onto the SDF-treated areas of dental tissues using bright field optical microscopy.

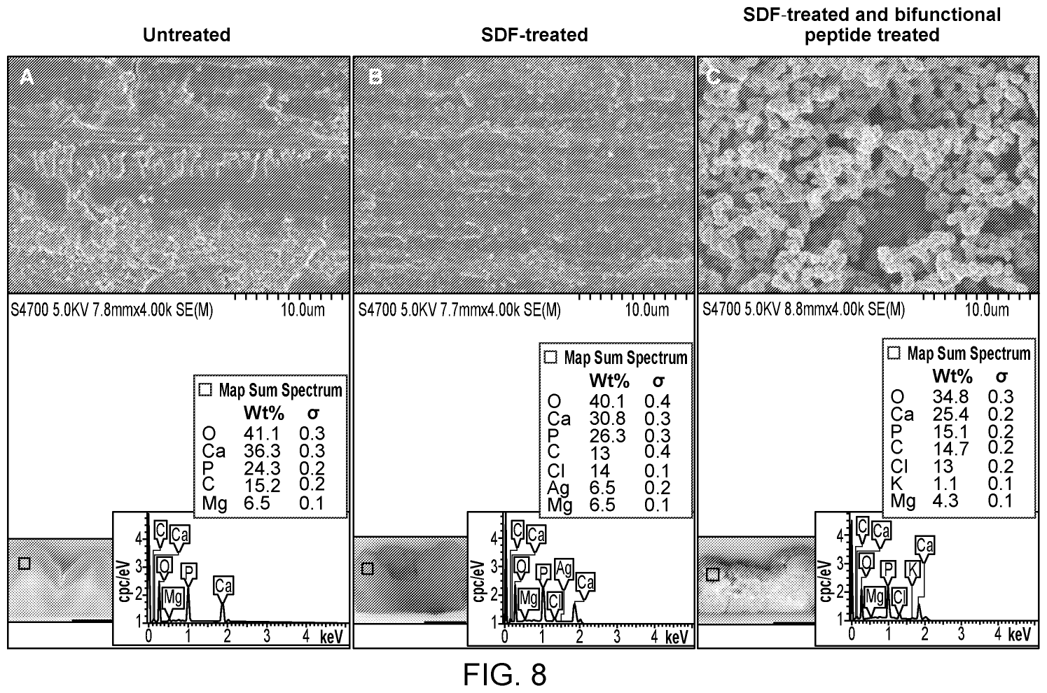

Treated dental tissues were analyzed using SEM for imaging and EDS for compositional analysis. Average Ca/P molar ratios were calculated for each slab of dental tissue from both SDF-treated and untreated regions of enamel and dentin, corresponding to SDF treatment of carious dental tissues, to assess mineral formation and compositional changes on the tooth samples in the presence and absence of the bifunctional peptide. SEM images with EDS spectra from representative regions of each treated dental tissue are shown in FIG. 8. Differences noted in the mineral morphology formed on the peptide mediated mineralized sample shown in FIG. 8 (top) were attributed to the presence of the mineral-mediating peptide MP. Average Ca/P ratios across treatment groups from both SDF-treated and untreated enamel and dentin regions are reported in Table 2.

TABLE 2
Averaged calculated Ca/P ratios of dental tissues in untreated
and SDF-treated regions in dentin and enamel across.

| | | | Mineralized Samples | |
|---|---|---|---|---|
| | | | (mineral layer formed on surface) | |
| | | SDF- | | SDF-treated |
| | Untreated | treated | | Peptide- |
| | Control | Control | SDF-treated | functionalized |
| Enamel | 1.375 | 1.447 | 1.092 | 1.338 |
| Dentin | 1.405 | 1.419 | 1.066 | 1.291 |
| SDF-stained | n/a | 1.540 | 1.100 | 1.257 |
| enamel | | | | |
| SDF-stained | n/a | 1.546 | 1.137 | 1.238 |
| dentin | | | | |

The average Ca/P ratios presented in Table 2 allow for direct comparisons between two sets of sample pairs, 1) untreated control and SDF-treated control, and 2) mineralized dental tissue slab with and without bifunctional peptide. The formed mineral layer on dental tissue slabs leads to differences in the CaP composition across the two groups, as the first group of data is directly formed on the dental tissue, rather than forming a new mineral layer. The SDF treatment resulted in slightly greater Ca/P ratios from untreated regions of the tooth compared to its untreated counterpart, with an increase in the regions from the enamel tissue. However, in the treated regions of the SDF-treated control dental tissue, a significantly greater Ca/P ratio was demonstrated for both SDF-treated enamel and dentin regions, as compared to untreated regions of the same sample and from the untreated control samples. A 2021 study investigated the effects of SDF on carious primary teeth regions and found SDF treatment promoted pathologic mineral by altering physiochemical properties of the tooth structures.⁸¹ Their detailed results included structural and elemental analysis of SDF-treated teeth which reported EDS results consistent with our findings.⁸¹ The increased Ca/P ratio in the SDF-treated control sample indicates that in these carious lesions of the tooth, the SDF treatment creates a more stable CaP material closer resembling a calcium-deficient apatite.^{44,59,76,82}

For the bifunctional peptide treated group, SDF-treated dental tissues were functionalized with either 0 μM or 50 μM bifunctional peptide prior to the mineralization protocol. Similar to what was observed with the Ag-coated samples (FIG. 6B), a significant increase in Ca/P ratio was observed in the sample functionalized with the bifunctional peptide. On average, the Ca/P ratios from the mineralized layer observed in both the peptide-treated and SDF-treated tissue as well as the SDF-only treated tissues, were about 0.1-0.15 less than what was obtained for the untreated and peptide-functionalized Ag-coated silica samples. The presence of calcium phosphate rich minerals in the slabs of dental tissue may alter the availability of free calcium and phosphate in the enzyme-mediated mineralization reaction. However, this same trend was noted, in the presence of the MP peptide where the Ca/P ratio was significantly greater than samples not functionalized with peptide, a finding confirming biomineralization activity of the peptide. Differences in the mineralization may also be attributed to the anchoring effect of the AgBP peptide being bound to the SDF-treated regions of the dental tissue.

### REFERENCES

1. Balakrishnan, M.; Simmonds, R. S.; Tagg, J. R. Dental caries is a preventable infectious disease. Aust Dent J 2000, 45, 235-245.
2. Caufield, P. W.; Li, Y.; Dasanayake, A. Dental caries: an infectious and transmissible disease. Compend Cantin Educ Dent 2005, 26, 10-16.
3. Chu, C.; Lo, E. Promoting caries arrest in children with silver diamine fluoride: a review. Oral health & preventive dentistry 2008, 6.
4. Yee, R.; Holmgren, C.; Mulder, J.; Lama, D.; Walker, D.; van Palenstein Helderman, W. Efficacy of silver diamine fluoride for arresting caries treatment. Journal of dental research 2009, 88, 644-647.
5. Çolak, H.; Dulgergil, Ç. T.; Dalli, M.; Hamidi, M. M. Early childhood caries update: A review of causes, diagnoses, and treatments. Journal of natural science, biology, and medicine 2013, 4, 29.
6. Dye, B. A.; Hsu, K.-L. C.; Afful, J. Prevalence and measurement of dental caries in young children. Pediatric dentistry 2015, 37, 200-216.
7. Kassebaum, N. J.; Bernabe, E.; Dahiya, M.; Bhandari, B.; Murray, C. J.; Marcenes, W. Global burden of untreated caries: a systematic review and metaregression. J Dent Res 2015, 94, 650-658, doi:10.1177/0022034515573272.
8. Gao, S. S.; Zhao, I. S.; Hiraishi, N.; Duangthip, D.; Mei, M.; Lo, E.; Chu, C. Clinical trials of silver diamine fluoride in arresting caries among children: a systematic review. JDR Clinical & Translational Research 2016, 1, 201-210.
9. Anil, S.; Anand, P. S. Early Childhood Caries: Prevalence, Risk Factors, and Prevention. Front Pediatr 2017, 5, 157-157, doi:10.3389/fped.2017.00157.
10. Institute of Medicine (U. S.). Committee on an Oral Health Initiative. Advancing oral health in America; National Academies Press: Washington, D.C., 2011; pp. xviii, 248 p.
11. Albino, J. a. D., B. A. Oral Health In America: Advances and Challenges. 2021.
12. Dye, B. A.; Albino, J.; D'Souza, R. N. Oral health problems are global and need to be addressed in the USA. Lancet 2022, 399, 127-128, doi:10.1016/S0140-6736(21)02842-7.
13. Shiboski, C. H.; Gansky, S. A.; Ramos-Gomez, F.; Ngo, L.; lsman, R.; Pollick, H. F. The association of early childhood caries and race/ethnicity among California preschool children. J Public Health Dent 2003, 63, 38-46.
14. Ramos-Gomez, F. J.; Crystal, Y. O.; Ng, M. W.; Crall, J. J.; Featherstone, J. D. B. Pediatric dental care: prevention and management protocols based on caries risk assessment. J Calif Dent Assoc 2010, 38, 746-761.
15. Jain, M.; Namdev, R.; Bodh, M.; Dutta, S.; Singhal, P.; Kumar, A. Social and behavioral determinants for early childhood caries among preschool children in India. Journal of dental research, dental clinics, dental prospects 2015, 9, 115.
16. Horowitz, H. S. Research issues in early childhood caries. Community dentistry and oral epidemiology 1998, 26, 67-81.
17. Congiu, G.; Campus, G.; Luglie, P. F. Early childhood caries (ECC) prevalence and background factors: a review. Oral Health Prev Dent 2014, 12, 71-76.
18. Berkowitz, R. J. Causes, treatment and prevention of early childhood caries: a microbiologic perspective. Journal-Canadian Dental Association 2003, 69, 304-307.
19. Halve, S.; Braun, P.; Irvine, J. D.; Nadeau, K.; Schroth, R. J.; Bell, S. L.; Calac, D. J.; Empey, A.; Nadeau, K. J.; Oski, J. A. Early childhood caries in indigenous communities. Pediatrics 2021, 147.
20. Casamassimo, P. S.; Thikkurissy, S.; Edelstein, B. L.; Maiorini, E. Beyond the dmft: the human and economic cost of early childhood caries. The Journal of the American Dental Association 2009, 140, 650-657.
21. Chu, C.; Lo, E.; Lin, H. Effectiveness of silver diamine fluoride and sodium fluoride varnish in arresting dentin caries in Chinese pre-school children. Journal of dental research 2002, 81, 767-770.
22. Marinho, V. Cochrane reviews of randomized trials of fluoride therapies for preventing dental caries. European Archives of Paediatric Dentistry 2009, 10, 183-191.
23. Hiiri, A.; Ahovuo-Saloranta, A.; Nordblad, A.; Makela, M. Pit and fissure sealants versus fluoride varnishes for preventing dental decay in children and adolescents. Cochrane Database of Systematic Reviews 2010.
24. Rosenblatt, A.; Stamford, T.; Niederman, R. Silver diamine fluoride: a caries “silver-fluoride bullet”. Journal of dental research 2009, 88, 116-125.
25. Horst, J. A.; Ellenikiotis, H.; Milgrom, P. L. UCSF Protocol for Caries Arrest Using Silver Diamine Fluoride: Rationale, Indications and Consent. J Calif Dent Assoc 2016, 44, 16-28.
26. Zhao, I. S.; Gao, S. S.; Hiraishi, N.; Burrow, M. F.; Duangthip, D.; Mei, M. L.; Lo, E. C.-M.; Chu, C.-H. Mechanisms of silver diamine fluoride on arresting caries: a literature review. International dental journal 2018, 68, 67-76.
27. Crystal, Y. O.; Niederman, R. Silver diamine fluoride treatment considerations in children's caries management. Pediatric dentistry 2016, 38, 466-471.
28. Contreras, V.; Toro, M. J.; Elias-Boneta, A. R.; Encarnacion-Burgos, M. A. Effectiveness of silver diamine fluoride in caries prevention and arrest: a systematic literature review. General dentistry 2017, 65, 22.
29. Cannon, A. B. Value of Silver Arsphenamie in the Treatment of Early Syphilis: Conclusions based on a Study of 104 Cases. Journal of the American Medical Association 1934, 102, 268-273, doi:10.1001/ja ma.1934. 02750040010003.
30. Nizami, M. Z. I.; Xu, V. W.; Yin, I. X.; Yu, O. Y.; Chu, C. H. Metal and Metal Oxide Nanoparticles in Caries Prevention: A Review. Nanomaterials (Basel}2021, 11, doi:10.3390/nanol1123446.
31. Seto, J.; Horst, J. A.; Parkinson, D. Y.; Frachella, J. C.; DeRisi, J. L. Enhanced Tooth Structure Via Silver Microwires Following Treatment with 38 Percent Silver Diamine Fluoride. Pediatr Dent 2020, 42, 226-231.
32. Milgrom, P.; Horst, J. A.; Ludwig, S.; Rothen, M.; Chaffee, B. W.; Lyalina, S.; Pollard, K. S.; DeRisi, J. L.; Mancl, L. Topical silver diamine fluoride for dental caries arrest in preschool children: A randomized controlled trial and microbiological analysis of caries associated microbes and resistance gene expression. J Dent 2018, 68, 72-78, doi:10.1016/j.jdent.2017.08.015.
33. Fung, H.; Wong, M. C.; Lo, E. C.; Chu, C. Arresting early childhood caries with silver diamine fluoride—a literature review. Journal of Oral Hygiene and Health 2013.
34. Horst, J. Silver fluoride as a treatment for dental caries. Advances in Dental Research 2018, 29, 135-140.
35. Peng, J.-Y.; Botelho, M.; Matinlinna, J. Silver compounds used in dentistry for caries management: a review. Journal of dentistry 2012, 40, 531-541.
36. Duangthip, D.; Jiang, M.; Chu, C. H.; Lo, E. C. Non-surgical treatment of dentin caries in preschool children-systematic review. BMC oral health 2015, 15, 1-10.
37. Seifo, N.; Cassie, H.; Radford, J. R.; Innes, N. P. T. Silver diamine fluoride for managing carious lesions: an umbrella review. BMC Oral Health 2019, 19, 145, doi:10.1186/s12903-019-0830-5.
38. Crystal, Y. O.; Janal, M. N.; Hamilton, D. S.; Niederman, R. Parental perceptions and acceptance of silver diamine fluoride staining. The Journal of the American Dental Association 2017, 148, 510-518. e514.
39. Crystal, Y. O.; Niederman, R. Evidence-based dentistry update on silver diamine fluoride. Dental Clinics 2019, 63, 45-68.
40. Ko, A. K.; Matsui, N.; Nakamoto, A.; Ikeda, M.; Nikaido, T.; Burrow, M. F.; Tagami, J. Effect of silver diammine fluoride application on dentin bonding performance. Dental materials journal 2020, 39, 407-414, doi:10.4012/dm;j.2019-057.
41. Tamerler, C.; Sarikaya, M. Genetically designed peptide-based molecular materials. ACS nano 2009, 3, 1606-1615, doi:10.1021/nn900720g.
42. Hnilova, M.; So, C. R.; Oren, E. E.; Wilson, B. R.; Kacar, T.; Tamerler, C.; Sarikaya, M. Peptide directed co-assembly of nanoprobes on multimaterial patterned solid surfaces. Soft Matter 2012, 8, 4327-4334.
43. Hnilova, M.; Karaca, B. T.; Park, J.; Jia, C.; Wilson, B. R.; Sarikaya, M.; Tamerler, C. Fabrication of hierarchical hybrid structures using bio-enabled layer-by-layer self-assembly. Biotechnology and bioengineering 2012, 109, 1120-1130.
44. Gungormus, M.; Oren, E. E.; Horst, J. A.; Fong, H.; Hnilova, M.; Somerman, M. J.; Snead, M. L.; Samudrala, R.; Tamerler, C.; Sarikaya, M. Cementomimetics constructing a cementum-like biomineralized microlayer via amelogenin-derived peptides. International Journal of Oral Science 2012, 4, 69-77, doi:10.1038/ijos.2012.40.
45. Ye, Q.; Spencer, P.; Yuca, E.; Tamerler, C. Engineered peptide repairs defective adhesive-dentin interface. Macromol Mater Eng 2017, 302, doi:10.1002/mame.201600487.
46. Wisdom, C.; Chen, C.; Yuca, E.; Zhou, Y.; Tamerler, C.; Snead, M. L. Repeatedly applied peptide film kills bacteria on dental implants. JOM 2019, 71, 1271-1280.
47. Xie, S.-X.; Boone, K.; VanOosten, S. K.; Yuca, E.; Song, L.; Ge, X.; Ye, Q.; Spencer, P.; Tamerler, C. Peptide Mediated Antimicrobial Dental Adhesive System. Appl Sci-Basel 2019, 9, 557.
48. Yuca, E.; Tamerler, C. Self-Assembled Recombinant Proteins on Metallic Nanoparticles as Bimodal Imaging Probes. JOM 2019, 71, 1281-1290.
49. Fischer, N. G.; Miinchow, E. A.; Tamerler, C.; Bottino, M. C.; Aparicio, C. Harnessing biomolecules for bioinspired dental biomaterials. Journal of Materials Chemistry B 2020, 8, 8713-8747.
50. Xie, S.-X.; Song, L.; Yuca, E.; Boone, K.; Sarikaya, R.; VanOosten, S. K.; Misra, A.; Ye, Q.; Spencer, P.; Tamerler, C. Antimicrobial peptide-polymer conjugates for dentistry. ACS applied polymer materials 2020, 2, 1134-1144.
51. Yuca, E.; Xie, S.-X.; Song, L.; Boone, K.; Kamathewatta, N.; Woolfolk, S. K.; Elrod, P.; Spencer, P.; Tamerler, C. Reconfigurable Dual Peptide Tethered Polymer System Offers a Synergistic Solution for Next Generation Dental Adhesives. International Journal of Molecular Sciences 2021, 22, 6552.
52. Sarikaya, R.; Song, L.; Yuca, E.; Xie, S.-X.; Boone, K.; Misra, A.; Spencer, P.; Tamerler, C. Bioinspired multifunctional adhesive system for next generation bio-additively designed dental restorations. Journal of the mechanical behavior of biomedical materials 2021, 113, 104135.
53. Hnilova, M.; Liu, X.; Yuca, E.; Jia, C.; Wilson, B.; Karatas, A. Y.; Gresswell, C.; Ohuchi, F.; Kitamura, K.; Tamerler, C. Multifunctional protein-enabled patterning on arrayed ferroelectric materials. ACS applied materials & interfaces 2012, 4, 1865-1871, d0i:10.1021/am300177t.
54. VanOosten, S. K.; Yuca, E.; Karaca, B. T.; Boone, K.; Snead, M. L.; Spencer, P.; Tamerler, C. Biosilver nanoparticle interface offers improved cell viability. Surface Innovations 2016, 4, 121-132, doi:10.1680/jsuin.16.00010.
55. Gibson, C. W.; Yuan, Z. A.; Hall, B.; Longenecker, G.; Chen, E.; Thyagarajan, T.; Sreenath, T.; Wright, J. T.; Decker, S.; Piddington, R.; et al. Amelogenin-deficient mice display an amelogenesis imperfecta phenotype. J Biol Chem 2001, 276, 31871-31875.
56. Snead, M. L. Amelogenin protein exhibits a modular design: implications for form and function. Connect Tissue Res 2003, 44 Suppl 1, 47-51.
57. Snead, M. L.; Zhu, D. H.; Lei, Y.; Luo, W.; Bringas, P. O., Jr.; Sucov, H. M.; Rauth, R. J.; Paine, M. L.; White, S. N. A simplified genetic design for mammalian enamel. Biomaterials 2011, 32, 3151-3157, doi:10.1016/j.biomaterials.2011.01.024.
58. Oren, E. E.; Tamerler, C.; Sahin, D.; Hnilova, M.; Seke, U. O.; Sarikaya, M.; Samudrala, R. A novel knowledge-based approach to design inorganic binding peptides. Bioinformatics 2007.
59. Dogan, S.; Fong, H.; Yucesoy, D. T.; Cousin, T.; Gresswell, C.; Dag, S.; Huang, G.; Sarikaya, M. Biomimetic tooth repair: amelogenin-derived peptide enables in vitro remineralization of human enamel. ACS Biomaterials Science & Engineering 2018, 4, 1788-1796.
60. Zhou, W.; Peng, X.; Zhou, X.; Bonavente, A.; Weir, M. D.; Melo, M. A. S.; Imazato, S.; Oates, T. W.; Cheng, L.; Xu, H. H. K. Novel Nanocomposite Inhibiting Caries at the Enamel Restoration Margins in an In Vitro Saliva-Derived Biofilm Secondary Caries Model. International Journal of Molecular Sciences 2020, 21, 6369.
61. Karaca, B. T.; Meyer, J.; VanQOosten, S.; Richter, M.; Tamerler, C. Modular Peptide-Based Hybrid Nanoprobes for Bio-Imaging and Bio-Sensing. MRS Online Proceedings Library (OPL) 2014, 1621, 155-161.
62. Zhang, S.; Karaca, B. T.; VanOosten, S. K.; Yuca, E.; Mahalingam, S.; Edirisinghe, M.; Tamerler, C. Coupling infusion and gyration for the nanoscale assembly of functional polymer nanofibers integrated with genetically engineered proteins. Macromolecular rapid communications 2015, 36, 1322-1328.
63. Lamiable, A.; Thevenet, P.; Rey, J.; Vavrusa, M.; Derreumaux, P.; Tuffery, P. PEP-FOLD3: Faster de novo structure prediction for linear peptides in solution and in complex. Nucleic Acids Res 2016, 44, W449-454, doi:10.1093/nar/gkw329.
64. Chen, J. E.; Huang, C. C.; Ferrin, T. E. RRDistMaps: a UCSF Chimera tool for viewing and comparing protein distance maps. Bioinformatics 2015, 31, 1484-1486.
65. Wisdom, E. C.; Zhou, Y.; Chen, C.; Tamerler, C.; Snead, M. L. Mitigation of peri-implantitis by rational design of bifunctional peptides with antimicrobial properties. ACS biomaterials science & engineering 2019, 6, 2682-2695.
66. Edwards, C. M.; Ulapane, S. B.; Kamathewatta, N. J. B.; Ashberry, H. M.; Berrie, C. L. Fabrication and Growth Control of Metal Nanostructures through Exploration of Atomic Force Microscopy-Based Patterning and Electroless Deposition Conditions. The Journal of Physical Chemistry C 2020, 124, 25588-25601, doi:10.1021/acs.jpcc.0c08017.
67. Ulapane, S. B.; Kamathewatta, N. J. B.; Ashberry, H. M.; Berrie, C. L. Controlled Electroless Deposition of Noble Metals on Silicon Substrates Using Self-Assembled Monolayers as Molecular Resists To Generate Nanopatterned Surfaces for Electronics and Plasmonics. ACS Applied Nano Materials 2019, 2, 7114-7125, doi:10.1021/acsanm.9b01641.
68. Hall Sedlak, R.; Hnilova, M.; Grosh, C.; Fong, H.; Baneyx, F.; Schwartz, D.; Sarikaya, M.; Tamerler, C.; Traxler, B. Engineered Escherichia coli silver-binding periplasmic protein that promotes silver tolerance. Applied and environmental microbiology 2012, 78, 2289-2296.
69. Palafox-Hernandez, J. P.; Tang, Z.; Hughes, Z. E.; Li, Y.; Swihart, M. T.; Prasad, P. N.; Walsh, T. R.; Knecht, M. R. Comparative Study of Materials-Binding Peptide Interactions with Gold and Silver Surfaces and Nanostructures: A Thermodynamic Basis for Biological Selectivity of Inorganic Materials. Chemistry of Materials 2014, 26, 4960-4969, doi:10.1021/cm501529u.
70. Wriggers, W.; Chakravarty, S.; Jennings, P. A. Control of protein functional dynamics by peptide linkers. Peptide Science: Original Research on Biomolecules 2005, 80, 736-746.
71. Hnilova, M.; Khatayevich, D.; Carlson, A.; Oren, E. E.; Gresswell, C.; Zheng, S.; Ohuchi, F.; Sarikaya, M.; Tamerler, C. Single-step fabrication of patterned gold film array by an engineered multifunctional peptide. J Colloid Interf Sci 2012, 365, 97-102, doi:DOI 10.1016/j.jcis.2011.09.006.
72. Wisdom, C.; VanOosten, S. K.; Boone, K. W.; Khvostenko, D.; Arnold, P. M.; Snead, M. L.; Tamerler, C. Controlling the biomimetic implant interface: Modulating antimicrobial activity by spacer design. J Mol Eng Mater 2016, 4, doi:10.1142/5S2251237316400050.
73. Chen, X.; Zaro, J. L.; Shen, W.-C. Fusion protein linkers: Property, design and functionality. Advanced Drug Delivery Reviews 2013, 65, 1357-1369, doi:http://dx.doi.org/10.1016/j.addr.2012.09.039.
74. Tan, S. F.; Chee, S. W.; Lin, G. H.; Mirsaidov, U. Direct Observation of Interactions between Nanoparticles and Nanoparticle Self-Assembly in Solution. Accounts Chem Res 2017, 50, 1303-1312, doi:10.1021/acs.accounts.7b00063.
75. Yazici, H.; Habib, G.; Boone, K.; Urgen, M.; Utku, F. S.; Tamerler, C. Self-assembling antimicrobial peptides on nanotubular titanium surfaces coated with calcium phosphate for local therapy. Materials Science and Engineering: C 2019, 94, 333-343, doi:https://doi.org/10.1016/j.msec.2018.09.030.
76. Yucesoy, D. T. Peptide-guided Dental Tissue Regeneration for Oral Care. 2018.
77. LeGeros, R. Z.; LeGeros, J. P. Calcium phosphate bioceramics: past, present and future. In Proceedings of the Key Engineering Materials, 2003; pp. 3-10.
78. LeGeros, R. Z. Calcium phosphate-based osteoinductive materials. Chemical reviews 2008, 108, 4742-4753.
79. Gungormus, M.; Fong, H.; Kim, L W.; Evans, J. S.; Tamerler, C.; Sarikaya, M. Regulation of in vitro calcium phosphate mineralization by combinatorially selected hydroxyapatite-binding peptides. Biomacromolecules 2008, 9, 966-973, doi:10.1021/bm701037×.
80. Sheikh, Z.; Abdallah, M.-N.; Hanafi, A. A.; Misbahuddin, S.; Rashid, H.; Glogauer, M. Mechanisms of in vivo degradation and resorption of calcium phosphate based biomaterials. Materials 2015, 8, 7913-7925.
81. Sulyanto, R.; Kang, M.; Srirangapatanam, S.; Berger, M.; Candamo, F.; Wang, Y.; Dickson, J.; Ng, M.; Ho, S. Biomineralization of dental tissues treated with silver diamine fluoride. Journal of Dental Research 2021, 100, 1099-1108.
82. Mei, M.; Nudelman, F.; Marzec, B.; Walker, J.; Lo, E.; Walls, A.; Chu, C. Formation of fluorohydroxyapatite with silver diamine fluoride. Journal of dental research 2017, 96, 1122-1128.
83. Roberts, A.; Bradley, J.; Merkley, S.; Pachal, T.; Gopal, J.; Sharma, D. Does potassium iodide application following silver diamine fluoride reduce staining of tooth? A systematic review. Australian dental journal 2020, 65, 109-117.
84. Hamdy, D.; Giraki, M.; Abd Elaziz, A.; Badran, A.; Allam, G.; Ruettermann, S. Laboratory evaluation of the potential masking of color changes produced by silver diamine fluoride in primary molars. BMC Oral Health 2021, 21, 337, doi:10.1186/s12903-021-01697-8.
85. Frohlich, T. T.; Gindri, L. D. O.; Pedrotti, D.; Cavalheiro, C. P.; Soares, F. Z. M.; Rocha, R. d. O. Evaluation of the Use of Potassium Iodide Application on Stained Demineralized Dentin Under Resin Composite Following Silver Diamine Fluoride Application. Pediatric Dentistry 2021, 43, 57-61.
86. Spencer, P.; Ye, Q.; Misra, A.; Goncalves, S. d. P.; Laurence, J. Proteins, pathogens, and failure at the composite-tooth interface. Journal of dental research 2014, 93, 1243-1249.
87. Drummond, J. L. Degradation, fatigue, and failure of resin dental composite materials. Journal of dental research 2008, 87, 710-719.
88. Chisini, L. A.; Collares, K.; Cademartori, M. G.; de Oliveira, L. J. C.; Conde, M. C. M.; Demarco, F. F.; Correa, M. B. Restorations in primary teeth: a systematic review on survival and reasons for failures. International journal of paediatric dentistry 2018, 28, 123-139.
89. Elbahie, E.; Beitzel, D.; Mutluay, M. M.; Majd, H.; Yahyazadehfar, M.; Arola, D. Durability of adhesive bonds to tooth structure involving the DEJ. Journal of the mechanical behavior of biomedical materials 2018, 77, 557-565, doi:https://doi.org/10.1016/j.jmbbm.2017.10.001.
90. Spencer, P.; Ye, Q.; Song, L.; Parthasarathy, R.; Boone, K.; Misra, A.; Tamerler, C. Threats to adhesive/dentin interfacial integrity and next generation bio-enabled multifunctional adhesives. J Biomed Mater Res B Appl Biomater 2019, 107, 2673-2683, doi:10.1002/jbm.b.34358.
91. Tripathi, P.; Mengi, R.; Gajare, S. M.; Nanda, S. S.; Wani, S. A.; Kochhar, A. S. Evaluation of Remineralizing Capacity of P11-4, CPP-ACP, Silver Diamine Fluoride, and NovaMin: An In Vitro Study. The Journal of Contemporary Dental Practice 2021, 22, 357-360.
92. Yuca, E.; Karatas, A. Y.; Seker, U. O.; Gungormus, M.; Dinler-Doganay, G.; Sarikaya, M.; Tamerler, C. In vitro labeling of hydroxyapatite minerals by an engineered protein. Biotechnol Bioeng 2011, 108, 1021-1030, doi:10.1002/bit.23041.
93. Wu, X.; Mahalingam, S.; VanOosten, S. K.; Wisdom, C.; Tamerler, C.; Edirisinghe, M. New Generation of Tunable Bioactive Shape Memory Mats Integrated with Genetically Engineered Proteins. Macromol Biosci 2017, 17, doi:10.1002/mabi.201600270.

While certain embodiments have been illustrated and described, it should be understood that changes and modifications may be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations may be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which may of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A bifunctional peptide of amino acid sequence

(SEQ ID NO: 1)
SYEKSHSQAINTDRT-X₁-EQLGVRKELRGV

or a pharmaceutically acceptable salt thereof and/or a solvate thereof, wherein X₁ is absent or a spacer of 1, 2, 3, 4, 5, 6, 7, 8, 9, or 10 amino acid residues.
B. The bifunctional peptide of Paragraph A, wherein X₁ is selected from EAAAK (SEQ ID NO: 2), APA, GGG, PAPAP (SEQ ID NO: 5), or GSGGG (SEQ ID NO: 6).
C. The bifunctional peptide of Paragraph A or Paragraph B, wherein the bifunctional peptide is SYEKSHSQAINTDRTEAAAKEQLGVRKELRGV (SEQ ID NO: 9).
D. The bifunctional peptide of Paragraph A or Paragraph B, wherein the bifunctional peptide is SYEKSHSQAINTDRTEQLGVRKELRGV (SEQ ID NO: 10).
E. The bifunctional peptide of Paragraph A or Paragraph B, wherein the bifunctional peptide is SYEKSHSQAINTDRTAPAEQLGVRKELRGV (SEQ ID NO: 11).
F. The bifunctional peptide of Paragraph A or Paragraph B, wherein the bifunctional peptide is SYEKSHSQAINTDRTGGGEQLGVRKELRGV (SEQ ID NO: 12).
G. The bifunctional peptide of Paragraph A or Paragraph B, wherein the bifunctional peptide is SYEKSHSQAINTDRTPAPAPEQLGVRKELRGV (SEQ ID NO: 13).
H. The bifunctional peptide of Paragraph A or Paragraph B, wherein the bifunctional peptide is SYEKSHSQAINTDRTGSGGGEQLGVRKELRGV (SEQ ID NO: 14).
I. A composition comprising the bifunctional peptide of any one of Paragraphs A-H and a pharmaceutically acceptable carrier.
J. The composition of Paragraph I, wherein the composition comprises an effective amount of the bifunctional peptide for treating a dental surface.
K. The composition of Paragraph I or Paragraph J, wherein the bifunctional peptide is present at a concentration of about 20 μM to about 150 μM.
L. A method of treating a dental surface, the method comprising:
   administering silver diamine fluoride (SDF) to the dental surface; and
   after administering the SDF, administering an effective amount of a compound of any one of Paragraphs A-H to the dental surface.
M. The method of Paragraph L, wherein the dental surface is a dental enamel and/or dentin.
N. The method of Paragraph M, wherein the dental enamel and/or dentin comprises a carious region, a hypomineralized region, or both a carious region and a hypomineralized region.
O. The method of any one Paragraphs L-N, wherein administering SDF to the dental surface comprises administering a solution comprising SDF to the dental surface.
P. The method of Paragraph O, wherein the solution has an SDF concentration of about 38% w/v.
Q. The method of any one of Paragraphs L-P, wherein administering the effective amount of the compound comprises contacting the dental surface with the compound for about 1 minute to about 4 hours.
R. The method of any one of Paragraphs L-Q, wherein administering the effective amount of the compound comprises contacting the dental surface with the compound for about 2 hours to about 4 hours.
S. The method of any one of Paragraphs L-R, wherein administering the effective amount of the compound adds new mineral to the dental surface.
T. The method of any one of Paragraphs L-S, wherein administering the effective amount of the compound reduces visible discoloration on the dental surface resulting from administering the SDF.
U. The method of any one of Paragraphs L-T, further comprising, after administering the effective amount of the compound, applying an adhesive dental composite to the dental surface.
V. A method of treating a dental surface, the method comprising:
   administering silver diamine fluoride (SDF) to the dental surface; and
   after administering the SDF, administering an effective amount of a composition of any one of Paragraphs I-K to the dental surface.
W. The method of Paragraph V, wherein the dental surface is a dental enamel and/or dentin.
X. The method of Paragraph W, wherein the dental enamel and/or dentin comprises a carious region, a hypomineralized region, or both a carious region and a hypomineralized region.
Y. The method of any one Paragraphs V-X, wherein administering SDF to the dental surface comprises administering a solution comprising SDF to the dental surface.
Z. The method of Paragraph Y, wherein the solution has an SDF concentration of about 38% w/v.
AA. The method of any one of Paragraphs V-Z, wherein administering the effective amount of the composition comprises contacting the dental surface with the compound for about 1 minute to about 4 hours.
AB. The method of any one of Paragraphs V-AA, wherein administering the effective amount of the composition comprises contacting the dental surface with the compound for about 2 hours to about 4 hours.
AC. The method of any one of Paragraphs V-AB, wherein administering the effective amount of the composition adds new mineral to the dental surface.
AD. The method of any one of Paragraphs V-AC, wherein administering the effective amount of the composition reduces visible discoloration on the dental surface resulting from administering the SDF.
AE. The method of any one of Paragraphs V-AD, further comprising, after administering the effective amount of the composition, applying an adhesive dental composite to the dental surface.

Other embodiments are set forth in the following claims, along with the full scope of equivalents to which such claims are entitled.

1. A bifunctional peptide of amino acid sequence
(SEQ ID NO: 1)
SYEKSHSQAINTDRT-X₁-EQLGVRKELRGV
or a pharmaceutically acceptable salt thereof and/or a solvate thereof,
wherein X₁ is absent or a spacer of 1, 2, 3, 4, 5, 6, 7, 8, 9, or 10 amino acid residues.

2. The bifunctional peptide of claim 1, wherein X₁ is selected from EAAAK (SEQ ID NO: 2), APA, GGG, PAPAP (SEQ ID NO: 5), or GSGGG (SEQ ID NO: 6).

3. The bifunctional peptide of claim 1, wherein the bifunctional peptide is SYEKSHSQAINTDRTEAAAKEQLGVRKELRGV (SEQ ID NO: 9).

4. The bifunctional peptide of claim 1, wherein the bifunctional peptide is SYEKSHSQAINTDRTEQLGVRKELRGV (SEQ ID NO: 10).

5. The bifunctional peptide of claim 1, wherein the bifunctional peptide is SYEKSHSQAINTDRTAPAEQLGVRKELRGV (SEQ ID NO: 11).

6. The bifunctional peptide of claim 1, wherein the bifunctional peptide is SYEKSHSQAINTDRTGGGEQLGVRKELRGV (SEQ ID NO: 12).

7. The bifunctional peptide of claim 1, wherein the bifunctional peptide is SYEKSHSQAINTDRTPAPAPEQLGVRKELRGV (SEQ ID NO: 13).

8. The bifunctional peptide of claim 1, wherein the bifunctional peptide is SYEKSHSQAINTDRTGSGGGEQLGVRKELRGV (SEQ ID NO: 14).

9. A composition comprising the bifunctional peptide of claim 1 and a pharmaceutically acceptable carrier.

10. The composition of claim 9, wherein the composition comprises an effective amount of the bifunctional peptide for treating a dental surface.

11. The composition of claim 9, wherein the bifunctional peptide is present at a concentration of about 20 μM to about 150 μM.

12. A method of treating a dental surface, the method comprising:
administering silver diamine fluoride (SDF) to the dental surface; and
after administering the SDF, administering an effective amount of a compound of claim 1 to the dental surface.

13. The method of claim 12, wherein the dental surface is a dental enamel and/or dentin.

14. The method of claim 13, wherein the dental enamel and/or dentin comprises a carious region, a hypomineralized region, or both a carious region and a hypomineralized region.

15. The method of claim 12, wherein administering SDF to the dental surface comprises administering a solution comprising SDF to the dental surface.

16. The method of claim 15, wherein the solution has an SDF concentration of about 38% w/v.

17. The method of claim 12, wherein administering the effective amount of the compound comprises contacting the dental surface with the compound for about 1 minute to about 4 hours.

18. The method of claim 12, wherein administering the effective amount of the compound comprises contacting the dental surface with the compound for about 2 hours to about 4 hours.

19.-20. (canceled)

21. The method of claim 12, further comprising, after administering the effective amount of the compound, applying an adhesive dental composite to the dental surface.

22. A method of treating a dental surface, the method comprising:
administering silver diamine fluoride (SDF) to the dental surface; and
after administering the SDF, administering an effective amount of a composition of claim 9 to the dental surface.

23.-31. (canceled)
